(19) United States
(12) Patent Application Publication
(10) Pub. No.: US 2022/0034880 A1
(43) Pub. Date: Feb. 3, 2022

(54) HIGH SPECIFICITY AND SENSITIVITY IMMUNOSORBENT DIAGNOSTIC ASSAYS WITH SIMULTANEOUS RESOLUTION OF MULTIPLE ANTIBODY ISOTYPES

(71) Applicant: The Board of Trustees of the Leland Stanford Junior University, Standford, CA (US)

(72) Inventors: Irving L. Weissman, Stanford, CA (US); Michal Caspi Tal, Cupertino, CA (US)

(21) Appl. No.: 17/279,012

(22) PCT Filed: Oct. 1, 2019
(86) PCT No.: PCT/US2019/054130

§ 371 Date: Mar. 23, 2021

Related U.S. Application Data
(60) Provisional application No. 62/739,626, filed on Oct. 1, 2018.
(60) Provisional application No. 62/862,397, filed on Jun. 17, 2019.

(51) Int. Cl. G01N 33/569; C12N 1/20

(52) U.S. Cl. CPC G01N 33/56911; C12N 1/20; G01N 2800/26; G01N 2469/20; G01N 2333/20

(57) ABSTRACT

Compositions and methods are provided for diagnosis of infections. The patterns of antibody isotype, subtype and glycosylation provide for a signature pattern that can identify infective agents and patient response to infection. Patients likely to benefit from therapeutic intervention can be discriminated from patients that have a low probability of responsiveness. Therapies are also provided.

### CROSS REFERENCE

This application claims the benefit of United States Provisional Application No. 62/739,626, filed Oct. 1, 2018, U.S. Provisional Application No. 62/862,397, filed Jun. 17, 2019 which is incorporated herein by reference in its entirety.

### BACKGROUND

Clinical detection of pathogen infection requires either the direct detection of pathogen in a biological specimen from the infected individual or indirect detection of pathogen specific antibodies. For infections that are difficult to detect with direct pathogen testing, for example where pathogens are present at very low numbers or in difficult to sample tissues, detection of pathogen specific antibodies is a more reliable method for diagnostic testing.

Conventional diagnostics for the detection of pathogen specific antibodies often rely on enzyme-linked immunosorbent assay or western blotting, which utilize either pathogen lysate or specific pathogen proteins/peptides to bind to pathogen specific antibodies and complex them to the pathogen or pathogen protein for downstream detection. However these strategies have significant limitations. The use of pathogen lysate introduces non-specific interactions with antibodies against highly conserved internal proteins shared by whole classes of pathogens. On the other hand, using only one specific protein or peptide can make the detection specific to one particular subspecies, such that many infections are missed. Furthermore, conventional immunosorbent assays display proteins or peptides of proteins that are not in their natural conformation, as they would be on the surface of the pathogen.

Further, conventional indirect testing for pathogen specific antibodies typically measure only bulk IgG, or IgM and IgG, and can miss other major isotypes, for example IgD, IgM, IgA, IgE and IgG, which can be further subset into subclasses which further contain multiple allotypes. For example, isotype IgA also contains subtype IgA2 which contains allotype IgA2m3. Different allotypes and subclasses may elicit different downstream effector functions by the immune system.

Lyme disease is caused by the bacteria Borrelia burgdorferi, which can be present in very low numbers and is difficult to sample directly from tissues. Lyme disease therefore requires indirect testing based on antibody production by the infected person. However, current diagnostics for this purpose have shortcomings. After being infected with Lyme disease, more than half of people who are tested with current methods will test negative during the most critical early treatment window of the first few weeks following infection (see Branda et al. (2018)).

The current diagnostic method for detecting Borrelia infection is a 2-tiered protocol requiring either an enzyme-linked immunosorbent assay (ELISA) or indirect fluorescence antibody, followed (if reactive) by Western immunoblots for immunoglobulin M and immunoglobulin G. These assays use bacterial lysates, which fail to maintain key binding epitopes and through display of intracellular proteins needlessly introduce non-specificity. The Western immunoblot component further introduces subjective interpretation and low sensitivity in early infection.

Improved methods for detection of pathogens that present at very low numbers or in difficult to sample tissues are of great clinical interest. It is critical to develop and improve sensitive and accurate diagnostics for a deeper understanding of these diseases and to enable the design of targeted therapeutics. The present invention addresses this.

### PUBLICATIONS

Benach, J. L. et al., 1986. An IgE response to spirochete antigen in patients with lyme disease. Zentralblatt für Bakteriologie, Mikrobiologie and Hygiene. Series A: Medical Microbiology, Infectious Diseases, Virology, Parasitology, 263(1-2), pp. 127-132.
Bluth, M. H. et al., 2007. IgE Anti-Borrelia burgdorferi Components (p18, p31, p34, p41, p45, p60) and Increased Blood CD8+CD60+T Cells in Children with Lyme Disease. Scandinavian Journal of Immunology, 65(4), pp. 376-382.
Branda, J. A. et al., Advances in Serodiagnostic Testing for Lyme Disease Are at Hand. Clinical Infectious Diseases VIEWPOINTS•CID, 2018, p. 1133.
Irani, V. et al., 2015. Molecular properties of human IgG subclasses and their implications for designing therapeutic monoclonal antibodies against infectious diseases. Molecular Immunology, 67(2), pp. 171-182.
Tjernberg, I. et al., 2017. IgE reactivity to α-Gal in relation to Lyme borreliosis. PloS one, 12(9), p. e0185723.

### SUMMARY

Compositions and methods are provided for the analysis of antibody responses to infection and identification of an infectious pathogen in an individual. The methods allow simultaneous analysis of a plurality of pathogen-specific antibody isotypes in an infected individual. In the methods, a diagnostic bait displaying a plurality of pathogen proteins/epitopes (e.g., a diagnostic test pathogen or antigen array) is contacted with an antibody containing sample from an individual, including without limitation blood samples and derivatives thereof. The diagnostic bait is washed free of unbound antibodies, and stained with one or more isotype-specific or glycosylation-specific labeling reagents, which reagents are operably linked to a detectable moiety, e.g. metal, colorimetric, fluorophore, etc. The diagnostic bait, thus labeled, is analyzed for the level of pathogen-specific antibodies, and the isotype distribution of antibodies. The methods allow simultaneous analysis of a plurality of pathogen-specific antibody isotypes from an infected individual. The identification of the pathogen-specific antibodies and the nature of the immune response to the pathogen allows appropriate selection of therapy for the individual.

In one aspect, a method of characterizing an immune response to a pathogen by an individual is provided, the method comprising: a) collecting at least one antibody-containing sample from the individual; b) contacting said at least one antibody-containing sample from the individual with a diagnostic bait displaying a plurality of pathogen proteins; c) contacting the diagnostic bait with one or more isotype-specific or glycosylation-specific reagents, which reagents are operably linked to a detectable moiety; and d) analyzing the diagnostic bait for the presence of bound isotype-specific or glycosylation-specific reagents to determine the presence and type of pathogen-specific antibodies, wherein the presence and type is indicative of a pathogen infection and immune response.

In some embodiments, a method comprises monitoring the immune response to the pathogen by the individual for a period of time by repeating steps a)-d) at a plurality of time points. For example, a first antibody-containing sample can be collected from the individual at a first time point and a second antibody-containing sample can be collected from the individual at a later second time point, wherein detection of increased levels of one or more pathogen-specific antibodies in the second sample compared to the levels of the one or more pathogen-specific antibodies in the first sample indicates that the infection by the pathogen is worsening, and decreased levels of one or more pathogen-specific antibodies in the second sample compared to the levels of the one or more pathogen-specific antibodies in the first sample indicates that the infection by the pathogen is improving. Serial sampling can be used to detect differences in the immune response to the pathogen over time which reveal changes that are indicative of infection. Serial sampling may be especially useful when the pathogen levels in an individual are initially at very low levels that are difficult to detect, wherein serial sampling makes it easier to distinguish infected individuals from uninfected individuals than if samples are collected at only a single timepoint.

In some embodiments, methods further comprises monitoring the efficacy of a therapy for treating an infection by a pathogen, wherein the first antibody-containing sample is collected from the individual before the patient undergoes the therapy and the second antibody-containing sample is collected from the individual after the patient undergoes the therapy, wherein detection of increased levels of the one or more pathogen-specific antibodies in the second antibody-containing sample compared to the levels of the one or more pathogen-specific antibodies in the first antibody-containing sample indicates that the infection by the pathogen is worsening or not responding to the therapy, and decreased levels of the one or more pathogen-specific antibodies in the second antibody-containing sample compared to the levels of the one or more pathogen-specific antibodies in the first antibody-containing sample indicates that the infection by the pathogen is improving.

In some embodiments, the diagnostic bait is a diagnostic pathogen, e.g, an intact pathogen. The diagnostic pathogen may be a cellular pathogen, e.g. bacteria, fungus, protozoan, etc., including, for example, Spirochaetes, such as Borrelia burgdorferi; fungal pathogens, such as Aspergillus fumigatus, Aspergillus flavus; protozoans such as Toxoplasma gondii, Plasmodium falciparum; and the like. The diagnostic pathogen may be, for example, a clinical isolate or an environmental isolate, or derived from a cell line or cell culture.

In some embodiments, a diagnostic pathogen is genetically modified to express a fluorophore, including without limitation a fluorescent protein, for example, green fluorescent protein (GFP), red fluorescent protein (RFP), and analogs thereof, including EGFP, EYFP, mYFP, Citrine, ECFP, mCFP, Cerulean, EBFP, and the like.

In some embodiments a diagnostic pathogen is further genetically modified to eliminate expression of proteins and other epitopes that are highly conserved among pathogens, thereby reducing non-specific binding to the diagnostic pathogen. In some embodiments the conserved epitopes are present on cell surface proteins. In some embodiments the epitopes are present of cell surface proteins that are highly conserved in the class of pathogens, e.g. among flagellar bacteria; among Spirochaetes, etc. In some embodiments the highly conserved proteins are flagella, including without limitation the fliH and/or flil proteins of Borrelia. In some embodiments the diagnostic pathogen, after binding to antibodies in the patient sample and labeling with isotype-specific or glycosylation-specific reagents, is inactivated.

In some embodiments, the diagnostic bait is an antigen array comprising pathogen proteins or peptide epitopes.

In some embodiments the labeled diagnostic bait (e.g., pathogen or antigen array comprising pathogen proteins or peptide epitopes bound to isotype-specific or glycosylation-specific reagents comprising a detectable moiety) is analyzed by a method that allows simultaneous analysis of multiple parameters, which parameters may include the isotype distribution of patent antibodies bound to the diagnostic pathogen, e.g. IgM, IgG1, IgG2, IgG2a, IgG2b, IgG3, IgG4, IgA, IgE, etc.; the glycosylation distribution of antibodies bound to the diagnostic pathogen, the overall level of antibody binding, and the like. In some embodiments the analysis is performed by flow cytometry. In some embodiments the analysis is performed by mass cytometry or microscopy.

In some embodiments the results of simultaneously measuring and comparing the ratios between the different subtypes of antibodies produced are utilized to identify the infectious pathogen for selection of an appropriate therapeutic regimen, e.g. antibiotics appropriate for treating the infectious pathogen. The stage of infection may also be inferred by the isotype distribution, where increases in IgG subclass antibodies relative to IgM class antibodies is indicative of a more advanced infection, which staging can be utilized for selection of an appropriate therapeutic regimen, e.g. where additional agents, anti-inflammatory therapies, and the like may be required for a more chronic state of infection. Assessment of the infectious agent and immune response by a patient allows improved care, where patients classified according to responsiveness can be treated with an appropriate agent. In an embodiment, the method further comprises determining a treatment course for the subject based on the analysis.

Patients can be classified upon initial presentation of symptoms, and can be further monitored for status over the course of the disease to maintain appropriate therapy, or can be classified at any appropriate stage of disease progression. In some embodiments, the method further comprises treating the individual. In some embodiments the presence of pathogen-specific IgE allows for distinction in therapeutic intervention, where one or more of anti-IgE therapy, mast cell stabilizer, and an antihistamine is administered to the individual if the presence of pathogen-specific immunoglobulin E (IgE) antibodies is detected. In some embodiments the antihistamine is an H2 antagonist.

In an embodiment, the method is implemented on one or more computers.

In an embodiment, the subject is a human subject.

In an embodiment, the immune response of an individual to a vaccine is determined to test the protective response against the infectious pathogen. In such embodiments, the vaccine immunogen corresponds to an infectious pathogen.

In some embodiments, a method of diagnosing an individual with Lyme disease is provided, the method comprising: a) collecting at least one antibody-containing sample from the individual; b) contacting said at least one antibody-containing sample from the individual with a diagnostic bait displaying a plurality of Borrelia burgdorferi pathogen antigens; c) contacting the diagnostic bait with one or more isotype-specific or glycosylation-specific reagents, which reagents are operably linked to a detectable moiety; d) analyzing the diagnostic bait for the presence of bound isotype-specific or glycosylation-specific reagents to determine the presence and type of Borrelia burgdorferi pathogen-specific antibodies, wherein the presence and type is indicative of a Borrelia burgdorferi infection and an immune response to the Borrelia burgdorferi pathogen; and e) diagnosing the individual with Lyme disease if the presence of one or more Borrelia burgdorferi pathogen-specific antibodies is detected.

In some embodiments, the method further comprises treating the individual for Lyme disease if the presence of one or more Borrelia burgdorferi pathogen-specific antibodies is detected. Identifying the presence of Borrelia burgdorferi pathogen-specific IgE allows for distinction in therapeutic intervention. In some embodiments, one or more of anti-IgE therapy, mast cell stabilizer, and an antihistamine is administered to the individual if the presence of pathogen-specific immunoglobulin E (IgE) antibodies is detected. The antihistamine may inhibit one or more histamine receptors selected from the group consisting of H1, H2, H3, and H4. In some embodiments the antihistamine is an H2 antagonist. In some embodiments, the method further comprising depleting mast cells in the individual if the presence of Borrelia burgdorferi pathogen-specific IgE antibodies is detected. Mast cells can be depleted, for example, by administering anti-c-kit therapy alone or in combination with anti-CD47 therapy. In some embodiments, the method further comprises administering an antibiotic. In some embodiments, the method further comprises depleting IgE producing B cells in the individual if the presence of Borrelia burgdorferi pathogen-specific immunoglobulin E (IgE) antibodies is detected. In some embodiments, anti-IgE therapy comprises IgE blockade or linkage of IgE specific antibodies to a different isotype with beneficial effector functions.

In some embodiments, the method further comprises monitoring the immune response to the Borrelia burgdorferi pathogen by the individual for a period of time by repeating steps a)-d) at a plurality of time points. For example, a first antibody-containing sample can be collected from the individual at a first time point and a second antibody-containing sample can be collected from the individual at a later second time point, wherein detection of increased levels of one or more Borrelia burgdorferi pathogen-specific antibodies in the second sample compared to the levels of the one or more Borrelia burgdorferi pathogen-specific antibodies in the first sample indicates that the infection by the Borrelia burgdorferi pathogen is worsening, and decreased levels of one or more Borrelia burgdorferi pathogen-specific antibodies in the second sample compared to the levels of the one or more Borrelia burgdorferi pathogen-specific antibodies in the first sample indicates that the infection by the Borrelia burgdorferi pathogen is improving. In some embodiments, the method further comprises monitoring the efficacy of a therapy for treating Lyme disease, wherein the first antibody-containing sample is collected from the individual before the patient undergoes the therapy and the second antibody-containing sample is collected from the individual after the patient undergoes the therapy, wherein detection of increased levels of the one or more Borrelia burgdorferi pathogen-specific antibodies in the second antibody-containing sample compared to the levels of the one or more Borrelia burgdorferi pathogen-specific antibodies in the first antibody-containing sample indicates that the Lyme disease is worsening or not responding to the therapy, and decreased levels of the one or more Borrelia burgdorferi pathogen-specific antibodies in the second antibody-containing sample compared to the levels of the one or more Borrelia burgdorferi pathogen-specific antibodies in the first antibody-containing sample indicates that the Lyme disease is improving.

In another embodiment, a Borrelia burgdorferi diagnostic pathogen is provided for use in the methods described herein.

In another embodiment, a kit is provided comprising a Borrelia burgdorferi diagnostic pathogen and one or more isotype-specific or glycosylation-specific reagents for detecting Borrelia burgdorferi pathogen-specific antibodies. In some embodiments, the kit comprises IgE-specific reagents for detecting Borrelia burgdorferi pathogen-specific IgE antibodies. In some embodiments, the kit further comprises one or more of an antihistamine, mast cell stabilizer, or an anti-IgE therapeutic agent.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 shows a schematic representation of a diagnostic immunosorbent assay. Borrelia burgdorferi genetically modified to express GFP (Bb-GFP) are incubated with serum from infected or uninfected hosts. These Bb-specific antibodies are then probed with a panel of fluorescently labeled isotype-specific secondary antibodies. These Bb are then assessed by flow cytometry for bacterial killing (loss of GFP), for size of aggregate formation, and for levels of various antibody binding to spirochetes.

FIG. 2A-2E show representative analyses of Borrelia-specific immune responses, and how they are impacted by infection conditions. FIG. 2A. Ankle swelling at peak inflammation. FIG. 2B. Titer of Borrelia specific antibody. FIG. 2C. Graph of antibody titer v. ankle swelling. FIG. 2D-2E. Comparison of serum sources for binding of IgG2a (FIG. 2D) and IgE (FIG. 2E).

FIG. 3A-3G provide time course analysis of antibody responses to infection. FIG. 3A. Change in antibody titers over 2 weeks post infection. FIG. 3B. IgG1 analysis 2 weeks post-infection. FIG. 3C. IgG2a analysis 2 weeks post-infection. FIG. 3D. IgM analysis 2 weeks post-infection. FIG. 3E. IgE analysis 2 weeks post-infection. FIG. 3F. Serum induced bacterial agglutination as assessed by FACS analysis 2 weeks post infection. FIG. 3G. FACS analysis of the size of the Bb-GFP.

FIGS. 4A-4B show IgE antibody responses to Bb infection are detectable 7 days post-infection in C3H mice but not in C57BL/6 mice. FIG. 4A shows results for C57BL/6 and C3H/HeJ mice infected with 10⁵ or 10 Bb-GFP. Serum was collected 7 days post-infection. A diagnostic immunoassay was performed as described in FIG. 1. FIG. 4B shows the percentage IgE bound at 1 week post-infection. IgE was detected in C3H mice but not in C57Bl/6 mice at this time point.

FIG. 5 shows that antihistamine treatment with Cimetidine, an H2 histamine receptor antagonist, reduces ankle swelling and IgE binding. Two weeks post-infection with 10⁶ Bb-GF, infected mice were given 2 mg/mL. Cimetidine in their drinking water. Peak ankle swelling was measured at day 49 PI (right) and a diagnostic immunoassay was performed on week 6 post-infection. IgE binding levels to Bb across different conditions are shown on the left.

FIGS. 6A-6C show antibody and Bb-immune complexes form when cultured Bb is exposed to serum from infected animals and is distinguishable from bactericidal antibody killing of Bb. Cultured Bb-GFP was incubated with serum from uninfected or infected animals as described in FIG. 1. By examining the fluorescence of the GFP in comparison to the size (Forward Scatter—FSC), it is possible to distinguish between single spirochetes, clumps, and antibody-induced Bb-immune complexes (denoted “super clumps”), as well as which bacteria still express or no longer express GFP. FIG. 6A shows 10⁶/100 Bb incubated overnight in BSK-H media with 6% rabbit serum (Sigma), with or without 10 μl of serum from mice that are either uninfected, or have been infected for the indicated amount of time. Representative microscopy is shown with Bb-GFP, IgE, and IgM. FIG. 6B shows graphs of dead bacteria (GFP-negative). FIG. 6C shows super clumps (Bb-immune complexes),

FIG. 7A-7D provides analysis of antibody binding of the indicated type to uninfected red blood cells compared to malaria (Plasmodium berghei ANKA (Pb-A)) infected red blood cells.

FIG. 8A-8D provides analysis of antibody binding of the indicated type with serum from Aspergillus fumigatus infected mice.

FIG. 9. HA-tagged recombinant Borrelia proteins (p66, p16s, and OspA) were immobilized on Ni-NTA beads and then incubated with serum from mice day 28 post infection with Borrelia burgdorferi and then probed for antibody isotypes and subtypes bound. IgG1 by IgE is shown for each protein type. OspA showed no IgE binding and low IgG1 binding while p66 and p16s both had efficient IgG1 and IgE binding, demonstrating that secondary antibody profiling can also be done on bead or chip protein or peptide arrays.

FIG. 10. IgE specific antibody responses to Bb infection are detectable 7 days post-infection in C3H mice but not in C57BL/6 mice. C57BL/6 and C3H/HeJ mice were infected intraperitoneal (IP) with 10⁵ Bb-GFP and serum was collected at indicated time points post-infection. A diagnostic immunoassay was performed as described in FIG. 1 and levels of IgE and IgG2a are shown.

FIG. 11. Mast cell degranulation exacerbates swelling. C3H/HeJ mice were infected intraperitoneal (IP) with 10⁵ Bb-GFP and tibiotarsal joint swelling was measured over the course of infection. On day 24 post-infection mice were injected retro-orbital with an antibody against cKIT which triggers mast cell degranulation or isotype control antibody.

### DETAILED DESCRIPTION

These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Most of the words used in this specification have the meaning that would be attributed to those words by one skilled in the art. Words specifically defined in the specification have the meaning provided in the context of the present teachings as a whole, and as are typically understood by those skilled in the art. In the event that a conflict arises between an art-understood definition of a word or phrase and a definition of the word or phrase as specifically taught in this specification, the specification shall control.

It must be noted that, as used in the specification and the appended claims, the singular forms “a,” “an,” and “the” include plural referents unless the context clearly dictates otherwise.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

All publications and patent applications cited in this specification are herein incorporated by reference as if each individual publication or patent application were specifically and individually indicated to be incorporated by reference.

Pathogen. As used herein, the term refers to an infectious organism, for example a bacteria, fungus, protozoan, virus, etc, that replicates in a host animal and thereby causes disease. In some embodiments the pathogen is a cellular pathogen, i.e. other than a virus, e.g. bacteria, single-celled fungi, protozoans, etc.

Pathogenic species may be bacteria, virus, protozoan parasites, fungal species, etc. Bacteria include Borrelia sp., Brucella sp., Treponema sp., Mycobacterium sp., Listeria sp., Legionella sp., Helicobacter sp, Streptococcus sp, Neisseria sp, Clostridium sp, Staphylococcus sp. or Bacillus sp.; including without limitation Treponema pallidum, Mycobacterium tuberculosis, Mycobacterium leprae, Listeria monocytogenes, Legionella pneumophila, Helicobacter pylori, Streptococcus pneumoniae, Neisseria meningitis, Clostridium novyi, Clostridium botulinum, Staphylococcus aureus, Bacillus anthracis, etc.

Parasite pathogens include Trichomonas, Toxoplasma, Giardia, Cryptosporidium, Plasmodium, Leishmania, Trypanosoma, Entamoeba, Schistosoma, Filariae, Ascaria, Fasciola; including without limitation Trichomonas vaginalis, Toxoplasma gondii, Giardia intestinalis, Cryptosporidium parva, Plasmodium falciparum, Trypanosoma cruzi, Entamoeba histolytica, Giardia lamblia, Fasciola hepatica, etc.

A pathogen may be infectious in humans, or in non-human mammals and avians, e.g, livestock such as cattle, sheep, pigs, poultry pets such as dogs, cats, birds; laboratory test animals such as mice, rats, rodents, non-human primates, and the like. Infection may be localized or systemic, e.g. skin, oral cavity, digestive tract, aural, etc.

A spirochaete is a member of the phylum Spirochaetes which contains distinctive diderm (double-membrane) bacteria, most of which have long, helically coiled cells. Spirochaetes are distinguished from other bacterial phyla by the location of their flagella, sometimes called axial filaments, which run lengthwise between the bacterial inner membrane and outer membrane in periplasmic space. These cause a twisting motion which allows the spirochaete to move about.

Many organisms within the Spirochaetes phylum cause prevalent diseases. Pathogenic members of this phylum include Leptospira species, Borrelia burgdorferi. B. garinii, and B. afzelii, which cause Lyme disease, Borrelia recurrentis, and B. hermsii, which cause relapsing fever, Treponema pallidum subspecies which cause syphilis and yaws, Brachyspira pilosicoli and Brachyspira aalborgi, which cause intestinal spirochaetosis.

Tick-borne diseases. In some embodiments the methods analyze an individual sample for evidence of infection by tick-borne parasites. Such diseases and parasites include, without limitation, Anaplasmosis (HGA): bacterium Anaplasma phagocytophllum; Tick-borne relapsing fever: Borrelia hermsii, B. turicatae, or B. parkerii Colorado tick fever: Coltivirus, Powassan encephalitis: Powassan virus, Babesiosis: Babesia parasites, Rocky Mountain Fever: Rickettsia rickettsii, Ehrlichiosis (HME): Ehrlichiachaffeensis, E. ewingii, or E. muris eauclairensis. In such embodiments, each diagnostic pathogen may be uniquely labeled, and analysis may be simultaneously performed on a cocktail of diagnostic pathogens.

Borrelia burgdorferi sensu lato is a group of spirochetes belonging to the genus Borrelia in the family of Spirochaetaceae. The spirochete is transmitted between reservoir hosts by ticks of the family Ixodidae. Infection with B. burgdorferi in humans may cause Lyme disease, or Lyme borreliosis, which is the most common vector-borne disease in North America and Europe. More than 40 species have been described in the genus Borrelia. These include 20 Borrelia species within the B. burgdorferi sensu lato complex and more than 20 Borrelia species associated with relapsing fever. The genus Borrelia possesses certain genetic and phenotypic characteristics that are unique among prokaryotes. Borrelia cells are helical with dimensions of 0.2 to 0.5 μm by 10 to 30 μm, allowing them to be easily distinguished from other eubacteria based on the phenotypic features common for all spirochetes. Borrelia can also be differentiated from other pathogenic spirochetes such as treponemes and leptospires on the basis of morphological traits, including the wavelength of the cell coils, the presence or absence of terminal hooks, the shape of the cell poles, and the number of periplasmic flagella. However, it is almost impossible to phenotypically distinguish different species within the Borrelia genus. Therefore, the identification and differentiation of different Borrelia species and strains is largely dependent on analyses of their genetic characteristics or on serology.

Five of the named Borrelia species are regularly found in human patients. These causative agents of human Lyme disease are B. burgdorferi sensu stricto in North America and Europe, B. garinii, B. bavariensis, B. afzelii in Europe and Asia, and B. spielmanii in Europe. Typing systems such as those provided herein that accurately characterize species and strains within species are crucial for epidemiological, clinical, and evolutionary studies.

Lyme Disease. Lyme disease is a tick-transmitted infection caused by Borrelia burgdorferi. Early symptoms include an erythema migrans rash, which may be followed weeks to months later by neurologic, cardiac, or joint abnormalities. Diagnosis is primarily clinical in early-stage disease, but serologic testing by the methods described herein can help diagnose cardiac, neurologic, and rheumatologic complications that occur later in the disease. Treatment is with antibiotics such as doxycycline or ceftriaxone, and may involve additional agents in later stages of the disease.

Lyme disease is transmitted primarily by 4 Ixodes sp worldwide: Ixodes scapularis (the deer tick) in the northeastern and north central US, I. pacificus in the western US, I. ricinus in Europe, I. persulcatus in Asia. B. burgdorferi enters the skin at the site of the tick bite. After 3 to 32 days, the organisms migrate locally in the skin around the bite, spread via the lymphatics to cause regional adenopathy or disseminate in blood to organs or other skin sites. Initially, an inflammatory reaction (erythema migrans) occurs before significant antibody response to infection (serologic conversion).

Lyme disease has 3 stages: Early localized, early disseminated, and late. The early and late stages are usually separated by an asymptomatic interval. Erythema migrans (EM), the hallmark and best clinical indicator of Lyme disease, is the first sign of the disease. It occurs in at least 75% of patients, beginning as a red macule or papule at the site of the tick bite, usually on the proximal portion of an extremity or the trunk (especially the thigh, buttock, or axilla), between 3 and 32 days after a tick bite. The area expands, often with clearing between the center and periphery resembling a bull's eye, to a diameter ˜50 cm. Darkening erythema may develop in the center, which may be hot to the touch and indurated. Without therapy, EM typically fades within 3 to 4 wk.

Symptoms of early-disseminated disease begin days or weeks after the appearance of the primary lesion, when the bacteria spread through the body. Soon after onset, nearly half of untreated patients develop multiple, usually smaller annular secondary skin lesions without indurated centers. Cultures of biopsy samples of these secondary lesions have been positive, indicating dissemination of infection. Patients also develop a musculoskeletal, flu-like syndrome, consisting of malaise, fatigue, chills, fever, headache, stiff neck, myalgias, and arthralgias that may last for weeks. Because symptoms are often nonspecific, the diagnosis is frequently missed if EM is absent. Symptoms are characteristically intermittent and changing, but malaise and fatigue may linger for weeks. Some patients develop symptoms of fibromyalgia. Resolved skin lesions may reappear faintly, sometimes before recurrent attacks of arthritis, in late-stage disease.

Neurologic abnormalities develop in about 15% of patients within weeks to months of EM (generally before arthritis occurs), commonly last for months, and usually resolve completely. Most common are lymphocytic meningitis or meningoencephalitis, cranial neuritis, and sensory or motor radiculoneuropathies, alone or in combination. Myocardial abnormalities occur in about 8% of patients within weeks of EM. They include fluctuating degrees of atrioventricular block (1st-degree, Wenckebach, or 3rd-degree) and, rarely, myopericarditis with chest pain, reduced ejection fractions, and cardiomegaly.

In untreated Lyme disease, the late stage begins months to years after initial infection. Arthritis develops in about 60% of patients within several months, occasionally up to 2 yr, of disease onset (as defined by EM). Intermittent swelling and pain in a few large joints, especially the knees, typically recur for several years. Affected knees commonly are much more swollen than painful; they are often hot, but rarely red. Baker cysts may form and rupture. Malaise, fatigue, and low-grade fever may precede or accompany arthritis attacks. In about 10% of patients, knee involvement is chronic. Other late findings (occurring years after onset) include an antibiotic-sensitive skin lesion (acrodermatitis chronica atrophicans) and chronic CNS abnormalities, either polyneuropathy or a subtle encephalopathy with mood, memory, and sleep disorders.

Treatment alternatives may vary with stage of disease but typically include amoxicillin, doxycycline, and ceftriaxone. In late-stage disease, antibiotics eradicate the bacteria, relieving the arthritis in most people. However, individuals may have persistent arthritis even after the infection has been eliminated because of continued inflammation and may be further treated with anti-inflammatory agents.

Individuals in which Borrelia burgdorferi-specific IgE antibodies are detected, may be administered anti-IgE therapy, mast cell stabilizer, or an antihistamine such as, but not limited to, cimetidine, ranitidine, Benadryl, diphenhydramine, loratadine, doxepin, thioperamide, and clobenpropit. Treatment may also include depleting mast cells in individuals having elevated levels of Borrelia burgdorferi-specific IgE antibodies. Mast cells can be depleted, for example, by administering anti-c-kit therapy alone or in combination with anti-CD47 therapy.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. Antibodies may refer to pathogen-specific serum antibodies present in an infected individual; and may also find use as the isotype or glycosylation specific labeling agents.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Clothia et al., J. Mol. Biol. 186:651 (1985); Novotny and Haber, Proc. Natl. Acad. Sci. U.S.A. 82:4592 (1985)).

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody.

An “isolated” antibody is one which has been identified and separated and/or recovered from a component of its natural environment. Contaminant components of its natural environment are materials which would interfere with diagnostic or therapeutic uses for the antibody, and can include enzymes, hormones, and other proteinaceous or nonproteinaceous solutes. In some embodiments, the antibody will be purified (1) to greater than 75% by weight of antibody as determined by the Lowry method, and most preferably more than 80%, 90% or 99% by weight, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Mast cell stabilizing drugs inhibit the release of allergic mediators from mast cells and are used clinically, e.g. to prevent allergic reactions. Mast cells have a role in allergic diseases because of hypersensitive response to substances that induces an allergic reaction, for example the release of preformed chemical mediators such as histamine, synthesis of lipid mediators such as PGs and LTs, production of cytokines and chemokines, etc. Mast cell stabilizers may be used at conventional dosages to reduce undesirable mast cell activation.

The most commonly used mast cell stabilizer is disodium cromoglycate, which inhibits IgE-dependent mast cell activation. Natural product mast stabilizers include, for example, Luteolin; Diosmetin; Quercetin; Fisetin; Kaempferol; Ginkgetin; Silymarin; Scopletin; Scaporone; Artekeiskeanol; Selinidin; Cinnamic acid; Ellagic acid; Magnolol and honokiol; Resveratrol; Polydatin; Curcumin; Mangostin-α, -β and -γ; Parthenolide; Sesquiterpene lactones; Monoterpenes; Sinomenine; Indoline; Xestospongin; Theanine; etc. Biologic inhibitors also include, for example, complement-derived peptide C3a, and the C3a9 peptide derived therefrom. Other anti-allergic peptides have been identified, e.g. LVA, LSY, RVS, ETI, TDG, RVV and GFW, which inhibited antigen-stimulated release of β-hexosaminidase from RBL-2H3 cells.

Synthetic and semi-synthetic mast cell stabilizers are also known in the art. For example indanone sesquiterpenes have been modified, and include the indanone, pterosin Z. Synthetic stabilizers include, for example, Compound 13, R112, ER-27317, U63A05, WHI-131, Hypothemycin, Midostaurin (PKC412), CP99994, K1 Ro 20-1724, rolipram and Siguazodan, Fullerenes, Vacuolin-1, CMT-3, OR-1384, OR-1958, TLCK, TPCK, Bromoenol lactone (BEL), Cerivastatin, atorvastatin and fluvastatin, Nilotinib, etc.

The term antihistamine is given its normal usage, i.e. a class of drug that opposes the activity of histamine receptors in the body, which are subclassified according to the histamine receptor that they act upon. The two largest classes of antihistamines are H1-antihistamines and H2-antihistamines. H1-antihistamines work by binding to histamine H1 receptors in mast cells, smooth muscle, and endothelium in the body as well as in the tuberomammillary nucleus in the brain. H2-antihistamines bind to histamine H2 receptors in the upper gastrointestinal tract, primarily in the stomach.

The majority of H1-antihistamines are receptor antagonists. Clinically, H1-antihistamines are used to treat allergic reactions and mast cell-related disorders. Examples of H1 antagonists include: Acrivastine, Azelastine, Bilastine, Bromodiphenhydramine, Brompheniramine, Buclizine, Carbinoxamine, Cetirizine, Chlorodiphenhydramine, Chlorpheniramine, Clemastine, Cyclizine, Cyproheptadine, Desloratadine (Aeries), Dexbrompheniramine, Dexchlorpheniramine, Dimenhydrinate, Dimetindene, Diphenhydramine, Doxylamine, Ebastine, Embramine, Fexofenadine, Hydroxyzine, Levocabastine, Levocetirizine, Loratadine, Meclizine, Mirtazapine, Olopatadine, Orphenadrine, Phenindamine, Pheniramine, Phenyltoloxamine, Promethazine

Quetiapine, Rupatadine, Tripelennamine, Triprolidine. Inverse H1 agonists include, for examples, Levocetirizine, Desloratadine, Pyrilamine, etc.

H2-antihistamines include, for example, Cimetidine, Famotidine, Lafutidine, Nizatidine, Ranitidine, Roxatidine, Tiotidine, etc.

Unless specifically indicated to the contrary, the term “conjugate” as described and claimed herein is defined as a heterogeneous molecule formed by the covalent attachment of one or more antibody fragment(s) to one or more detectable moieties.

“Suitable conditions” shall have a meaning dependent on the context in which this term is used. That is, when used in connection with an antibody, the term shall mean conditions that permit an antibody to bind to its corresponding antigen. When used in connection with contacting an agent to a cell, this term shall mean conditions that permit an agent capable of doing so to enter a cell and perform its intended function. In one embodiment, the term “suitable conditions” as used herein means physiological conditions.

A “subject” or “patient” in the context of the present teachings is generally a mammal. Mammals other than humans can be advantageously used as subjects that represent animal models of inflammation. A subject can be male or female.

As used herein, the terms “detectable moiety”, “detection agent”, and “detectable label” are used interchangeably and refer to a molecule or substance capable of detection, including, but not limited to, fluorescers, chemiluminescers, chromophores, bioluminescent proteins, enzymes, enzyme substrates, enzyme cofactors, enzyme inhibitors, isotopic labels, semiconductor nanoparticles, dyes, metal ions, metal sols, ligands (e.g., biotin, streptavidin or haptens) and the like. The term “fluorescer” refers to a substance or a portion thereof which is capable of exhibiting fluorescence in the detectable range. Particular examples of labels which may be used in the practice of the invention include, but are not limited to, fluorescent proteins such as green fluorescent protein (GFP), red fluorescent protein (REP), enhanced GFP (EGFP), superfolder GFP (sfGFP), blue fluorescent protein (EBFP, EBFP2, Azurite, mKalama1), cyan fluorescent protein (ECFP; Cerulean, CyPet, mTurquoise2), yellow fluorescent protein and derivatives (YFP, Citrine, Venus, YPet), dsRed, eqFP611, Dronpa, TagRFPs, KFP, EosFP/IrisFP, and Dendra; fluorescent dyes including, but not limited to, a SYBR dye such as SYBR green and SYBR gold, a CAL Fluor dye such as CAL Fluor Gold 540, CAL Fluor Orange 560, CAL Fluor Red 590; CAL Fluor Red 610, and CAL Fluor Red 635, a Quasar dye such as Quasar 570, Quasar 670, and Quasar 705, an Alexa Fluor such as Alexa Fluor 350, Alexa Fluor 488, Alexa Fluor 546, Alexa Fluor 555, Alexa Fluor 594, Alexa Fluor 647, and Alexa Fluor 784, a cyanine dye such as Cy 3, Cy3.5, Cy5, Cy5.5, and Cy7, fluorescein, 2′, 4′, 5′, 7′-tetrachloro-4-7-dichlorofluorescein (TET), carboxyfluorescein (FAM); 6-carboxy-4′,5′-dichloro-2′,7′-dimethoxyfluorescein (JOE), hexachlorofluorescein (HEX); rhodamine, carboxy-X-rhodamine (ROX), tetramethyl rhodamine (TAMRA), FITC, dansyl, umbelliferone, dimethyl acridinium ester (DMAE), Texas red, luminol, and quantum dots; and enzymes such as alkaline phosphatase (AP), beta-lactamase, chloramphenicol acetyltransferase (CAT), adenosine deaminase (ADA), aminoglycoside phosphotransferase (neo^{r}, G418^{r}) dihydrofolate reductase (DHFR), hygromycin-B-phosphotransferase (HPH), thymidine kinase (TK), f-galactosidase (lacZ), and xanthine guanine phosphoribosyltransferase (XGPRT), beta-glucuronidase (gus), placental alkaline phosphatase (PLAP), and secreted embryonic alkaline phosphatase (SEAP). Enzyme tags are used with their cognate substrate. The terms also include chemiluminescent labels such as luminol, isoluminol, acridinium esters, and peroxyoxalate and bioluminescent proteins such as firefly luciferase, bacterial luciferase, Renilla luciferase, and aequorin. The terms also include isotopic labels, including radioactive and non-radioactive isotopes, such as; ³H, ²H, ¹²⁰I, ¹²³I, ¹²⁴I, ¹²⁵I, ¹³¹I, ³⁵S, ¹¹C, ¹³C, ¹⁴C, ³²P, ¹⁵N, ¹³N, ¹¹⁰In, ¹¹¹In, ¹⁷⁷Lu; ¹⁸F, ⁵²Fe, ⁵²Cu, ⁶⁴Cu, ⁶⁷Cu, ⁶⁷Ga; ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁵Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb, and ⁵³Sr. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g.; Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies; Inc.), encoded microparticles with colored bar codes (see e.g.; CellCard produced by Vitra Bioscience, vitrabio.com), glass microparticles with digital holographic code images (see e.g., CyVera microbeads produced by IIlumina (San Diego, Calif.), near infrared (NIR) probes, and nanoshells.

To “analyze” includes determining a set of values associated with a sample by measurement of a marker (such as, e.g., presence or absence of an antibody isotype) in the sample and comparing the measurement against measurement in a sample or set of samples from the same subject or other control subject(s). The markers of the present teachings can be analyzed by any of various conventional methods known in the art. To “analyze” can include performing a statistical analysis to, e.g., determine whether a subject is infected with a pathogen of interest, the stage of infection, and the like.

A “sample” in the context of the present teachings refers to any biological sample that is isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between cells, including gingival crevicular fluid, bone marrow, cerebrospinal fluid (CSF), saliva, mucous, sputum, semen, sweat, urine, or any other bodily fluids.

“Blood sample” can refer to whole blood or any fraction thereof, including blood cells, red blood cells, white blood cells or leucocytes, platelets, serum and plasma. Samples can be obtained from a subject by means including but not limited to venipuncture, excretion, ejaculation, massage, biopsy, needle aspirate, lavage, scraping, surgical incision, or intervention or other means known in the art.

A “dataset” is a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring by the methods described herein. The phrase also encompasses receiving a set of data, e.g., from a third party that has processed the sample to experimentally determine the dataset. Additionally, the phrase encompasses mining data from at least one database or at least one publication or a combination of databases and publications.

“Measuring” or “measurement” in the context of the present teachings refers to determining the presence, absence, quantity, amount, or effective amount of a substance, typically pathogen-specific antibodies, in a clinical or subject-derived sample, including the presence, absence, or concentration levels of such substances, and/or evaluating the values or categorization of a subject's clinical parameters based on a control.

Classification can be made according to predictive modeling methods that set a threshold for determining the probability that a sample belongs to a given class. The probability preferably is at least 50%, or at least 60% or at least 70% or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class, i.e. whether an infection is present, the stage of infection, and the like.

The predictive ability can be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC (area under the curve) of at least about 0.7, at least about 0.75, at least about 0.8, at least about 0.85, at least about 0.9, or higher.

As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about at least about 0.7, at least about 0.75, at least about 0.8, at least about 0.85, at least about 0.9, or higher.

Unless otherwise apparent from the context, all elements, steps or features of the invention can be used in any combination with other elements, steps or features.

General methods in molecular and cellular biochemistry can be found in such standard textbooks as Molecular Cloning; A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

The subject methods are used for diagnostic or therapeutic purposes. As used herein, the term “treating” is used to refer to both prevention of relapses, and treatment of pre-existing conditions. For example, the prevention of inflammatory disease that is a result of infection can be accomplished by administration of the agent at an earlier stage of infection, i.e. with highly sensitive methods. The treatment of ongoing disease, where the treatment stabilizes or improves the clinical symptoms of the patient, is of particular interest.

### Methods

Methods are provided for the analysis of antibody responses to infection and identification of an infectious pathogen in an individual. The methods allow simultaneous analysis of a plurality of pathogen-specific antibody isotypes. In the methods, a diagnostic bait displaying a plurality of pathogen antigens/epitopes, or an intact pathogen, is contacted with an antibody containing sample from an individual, including without limitation blood samples and derivatives thereof. The diagnostic bait is washed free of unbound antibodies, and stained with one or more isotype-specific or glycosylation-specific labeling reagents, which reagents are operably linked to a detectable moiety, e.g. metal, colorimetric, fluorophore, etc. The diagnostic bait, thus labeled, is analyzed for the level of pathogen-specific antibodies and the isotype distribution of antibodies. The identification of the pathogen-specific antibodies and the nature of the immune response to the pathogen allows appropriate selection of a therapy for the individual.

In some embodiments, the diagnostic bait is an intact live pathogen (i.e., diagnostic pathogen). The diagnostic pathogen may be genetically modified to express a fluorophore, including without limitation a fluorescent protein, for example green fluorescent protein (GFP); red fluorescent protein (RFP), and analogs thereof, including EGFP, EYFP, mYFP, Citrine, ECFP, mCFP, Cerulean, EBFP; and the like. The diagnostic pathogen may be the same species as the infectious pathogen, or may be a closely related species, e.g. the diagnostic pathogen may be one species in the Borrelia genus, but may be used to detect closely related Borrelia sp.

In some embodiments the diagnostic pathogen is further genetically modified to eliminate expression of proteins and other epitopes that are highly conserved among pathogens, thereby reducing non-specific binding to the diagnostic pathogen. In some embodiments the epitopes are present on cell surface proteins. In some embodiments the epitopes are present on cell surface proteins that are highly conserved in the class of pathogens, e.g, among flagellar bacteria; among spirochaetes, etc. In some embodiments the highly conserved proteins are flagellar proteins, including without limitation the fliH and/or flil proteins of Borrelia.

In some embodiments, the diagnostic pathogen is fixed prior to contacting with the isotype-specific and/or glycosylation-specific labeling reagents. For instance, in certain embodiments, a cellular pathogen may be fixed with one or more crosslinking agents such as formaldehyde, glutaraldehyde, or bifunctional linkers such as ethylene glycol bis(succinimidyl succinate (EGS); or fixed by dehydration with alcohols such as methanol or ethanol.

In other embodiments, the diagnostic bait is an antigen array displaying pathogen proteins or peptides. The antigen array can be contacted with an antibody containing sample from an individual, wherein pathogen-specific antibodies from the sample bind to the displayed epitopes on the proteins/peptides of the antigen array. Antigen arrays can be generated by immobilizing pathogen proteins and/or peptides on a solid support using methods well-known in the art. The solid support may include, for example, without limitation, a glass slide; plastic, metal, a gel, a membrane; silica, beads; or nanoparticles. Such antigen arrays can be designed to display a representative number of peptides or proteins from the pathogen proteome and, in particular, pathogenic proteins of interest that contribute to pathological inflammatory immune responses. In addition, the array may comprise proteins that are not derived from the pathogen for use as controls. For a discussion of methods of fabricating antigen arrays, see, e.g., Yuan et al. (2017) Methods Mol. Biol. 1654:271-227 and Robinson (2006) Curr. Opin. Chem. Biol. 10(1):67-72; herein incorporated by reference.

In some embodiments the labeled diagnostic bait is analyzed by a method that allows simultaneous analysis of multiple parameters, which parameters may include the isotype distribution of patent antibodies bound to the diagnostic pathogen, e.g. IgM, IgG1, IgG2, IgG2a, IgG2b; IgG3; IgG4; IgA, IgE, etc.; the glycosylation distribution of antibodies bound to the diagnostic pathogen, the overall level of antibody binding, and the like.

In the methods provided herein, a cocktail of labeling reagents may be used, where each is specific for an antibody isotype or glycosylation pattern. A cocktail may comprise 2, 3, 4, 5, 6, 7, 8, or more different labeling reagents. In some embodiments the labeling reagents are antibodies conjugated to a detectable moiety, which antibodies specifically bind to an isotype or glycosylation pattern of interest. In other embodiments the reagent is a labeled aptamer specific for an isotype, for example as described by Ma et al., (2013) Genet. Mol, Res. 12(2):1399-410, or as commercially available from Aptagen, human Immunoglobulin G (IgG) (Apt 8) (ID #44).

Each labeling reagent is typically labeled with a different label, e.g. fluorophore, metal, etc. A set of fluorescent labels is typically chosen such that they can be identified using FACS, e.g. FITC, BV650, evolve 655, BV605, K-Orange, eF450, PE-Cy7, PerCP-Cy5.5, PE, FITC/AF488, APC-eF780, AF700 and APC, etc. substantially equivalent fluorophores. Any combination of these labels may be used.

Alternatively, mass cytometry may be used. Mass cytometry is suitable for the simultaneous detection of many more than one labelling atom, permitting multiplex label detection e.g., at least 3, 4, 5, 10, 20, 30, 32, 40, 50 or even 100 different labelling atoms. Labelling atoms that can be used include any species that are detectable by ICP-MS and that are substantially absent from the unlabeled sample. In preferred embodiments, the labelling atoms are transition metals, such as the rare earth metals (the 15 lanthanides, plus scandium and yttrium). These 17 elements provide many different isotopes which can be easily distinguished by ICP-MS. A wide variety of these elements are available in the form of enriched isotopes e.g., samarium has 6 stable isotopes, and neodymium has 7 stable isotopes, all of which are available in enriched form. The 15 lanthanide elements provide at least 37 isotopes that have non-redundantly unique masses. Examples of elements that are suitable for use as labelling atoms include Lanthanum (La), Cerium (Ce), Praseodymium (Pr), Neodymium (Nd), Promethium (Pm), Samarium (Sm), Europium (Eu), Gadolinium, (Gd), Terbium (Tb), Dysprosium (Dy), Holmium (Ho), Erbium (Er), Thulium (Tm), Ytterbium (Yb), Lutetium (Lu), Scandium (Sc), and Yttrium (Y). In addition to rare earth metals, other metal atoms are suitable for detection by ICP-MS e.g., gold (Au), platinum (Pt), iridium (Ir), rhodium (Rh), bismuth (Bi), etc.

To permit differential detection of isotypes or glycosylation patterns, the labeling reagents should carry different labelling atoms such that their signals can be distinguished by ICP-MS. For instance, where ten different isotypes are being detected, ten different antibodies can be used, each of which carries a unique label, such that signals from the different antibodies can be distinguished.

Other analytic methods may include polymerase chain reaction (PCR) of DNA sequence barcoded antibodies. PCR is a technique to exponentially amplify a specific segment of DNA to generate many copies of a particular DNA sequence. Alternatively, sequence barcoded antibodies could be analyzed through sequencing the DNA barcodes. Sequencing is a process by which the order of nucleotides in a DNA sequence is determined.

Colorimetric or fluorescence labeling of antibodies could be detected through high dimensional, multi parameter microscopy which facilitates the detection of multiple different antibodies simultaneously or in sequence. This can be combined with DNA sequence barcoded antibodies for sequential imaging and quenching to allow for identification of anywhere from 2 to 50 (or potentially even more) different types of antibodies in a particular sample.

An immune response pattern to a pathogen can be generated from a biological sample using any convenient protocol, for example as described above. The readout can be a mean, average, median or the variance or other statistically or mathematically-derived value associated with the measurement. The marker readout information can be further refined by direct comparison with the corresponding reference or control pattern. A binding pattern can be evaluated on a number of points: to determine if there is a statistically significant change at any point in the data matrix; whether the change is an increase or decrease in the binding; whether the change is specific for one or more physiological states, and the like. The absolute values obtained for each marker under identical conditions will display a variability that is inherent in live biological systems and also reflects the variability inherent between individuals.

Following obtainment of the immune response pattern to a pathogen from the sample being assayed, the immune response pattern to a pathogen is compared with a reference or control profile to make a prognosis regarding the state and stage of infection of the patient from which the sample was obtained/derived. Typically a comparison is made with a sample or set of samples from an unaffected, normal source. Additionally, a reference or control pattern can be a signature pattern that is obtained from a sample of a patient known to be infected, and therefore can be a positive reference or control profile.

Patient infection and staging status can be assessed using clinical-based criteria. In order to identify subsets that are indicative of these stages and subsets, a statistical test will provide a confidence level for a change in the expression, titers or concentration of markers between the test and control profiles to be considered significant, where the control profile can be for responsiveness or non-responsiveness. The raw data can be initially analyzed by measuring the values for each marker, usually in duplicate, triplicate, quadruplicate or in 5-10 replicate features per marker.

A test dataset is considered to be different than a control dataset if one or more of the parameter values of the profile exceeds the limits that correspond to a predefined level of significance.

To provide significance ordering, the false discovery rate (FDR) can be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (see Tusher et al, (2001) PNAS 98, 5116-21, herein incorporated by reference). This analysis algorithm is currently available as a software “plug-in” for Microsoft Excel know as Significance Analysis of Microarrays (SAM). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value can be applied to the correlations between experimental profiles.

For SAM, Z-scores represent another measure of variance in a dataset, and are equal to a value of X minus the mean of X, divided by the standard deviation. A Z-Score tells how a single data point compares to the normal data distribution. A Z-score demonstrates not only whether a datapoint lies above or below average, but how unusual the measurement is. The standard deviation is the average distance between each value in the dataset and the mean of the values in the dataset.

Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pairwise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation.

The data can be subjected to non-supervised hierarchical clustering to reveal relationships among profiles. For example, hierarchical clustering can be performed, where the Pearson correlation is employed as the clustering metric. One approach is to consider a patient disease dataset as a “learning sample” in a problem of “supervised learning”. CART is a standard in applications to medicine (Singer (1999) Recursive Partitioning in the Health Sciences, Springer), which can be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

Other methods of analysis that can be used include logic regression. One method of logic regression Ruczinski (2003) Journal of Computational and Graphical Statistics 12:475-512. Logic regression resembles CART in that its classifier can be displayed as a binary tree. It is different in that each node has Boolean statements about features that are more general than the simple “and” statements produced by CART.

Another approach is that of nearest shrunken centroids (Tibshirani (2002) PNAS 99:6567-72). The technology is k-means-like, but has the advantage that by shrinking cluster centers, one automatically selects features (as in the lasso) so as to focus attention on small numbers of those that are informative. The approach is available as Prediction Analysis of Microarrays (PAM) software, a software “plug-in” for Microsoft Excel, and is widely used. Two further sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome. Several of these methods are based on the “R” software, developed at Stanford University, which provides a statistical framework that is continuously being improved and updated in an ongoing basis.

Other statistical analysis approaches including principle components analysis, recursive partitioning, predictive algorithms, Bayesian networks, and neural networks.

These statistical tools are applicable to all manner of serological data. A set of data that can be easily determined, and that is highly informative regarding detection of individuals with infections of different stages and responsiveness to therapy are provided. Also provided are databases of signature patterns for immune responsiveness to a pathogen. Such databases will typically comprise signature patterns of individuals having specific types and stages of immune responses, etc., where such profiles are as described above.

The analysis and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data can be used for a variety of purposes, such as patient monitoring, initial diagnosis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer can be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the immune response to a pathogen is monitored in the individual for a period of time by repeating the steps of the diagnostic method at a plurality of time points. For example, a first antibody-containing sample can be collected from the individual at a first time point and a second antibody-containing sample can be collected from the individual at a second time point (later), wherein detection of increased levels of one or more pathogen-specific antibodies in the second antibody-containing sample compared to the levels of the one or more pathogen-specific antibodies in the first antibody-containing sample indicates that the infection by the pathogen is worsening, and decreased levels of the one or more pathogen-specific antibodies in the second antibody-containing sample compared to the levels of the one or more pathogen-specific antibodies in the first antibody-containing sample indicates that the infection by the pathogen is improving. Serial sampling can be used to detect differences in the immune response to the pathogen over time which reveal changes that are indicative of infection. Serial sampling of antibody levels from an individual may be useful, especially in cases when the pathogen levels in an individual are initially at very low levels that are difficult to detect, wherein serial sampling makes it easier to distinguish infected individuals from uninfected individuals than if samples are collected at only a single timepoint.

In some embodiments, serial sampling is used for monitoring the efficacy of a therapy for treating an infection in a patient using the methods described herein. For example, a first antibody-containing sample can be collected from the individual before the patient undergoes the therapy and a second antibody-containing sample can be collected from the individual after the patient undergoes the therapy, wherein detection of increased levels of one or more pathogen-specific antibodies in the second antibody-containing sample compared to the levels of the one or more pathogen-specific antibodies in the first antibody-containing sample indicates that the infection by the pathogen is worsening or not responding to the therapy, and decreased levels of the one or more pathogen-specific antibodies in the second antibody-containing sample compared to the levels of the one or more pathogen-specific antibodies in the first antibody-containing sample indicates that the infection by the pathogen is improving.

The term “antibiotic” as used herein includes all commonly used bacteristatic and bactericidal antibiotics, usually those administered orally. Antibiotics include aminoglycosides, such as amikacin, gentamicin, kanamycin, neomycin, streptomycin, and tobramycin; cephalosporins, such as cefamandole, cefazolin, cephalexin, cephaloglycin, cephaloridine, cephalothin, cephapirin, and cephradine; macrolides, such as erythromycin and troleandomycin; penicillins, such as penicillin G, amoxicillin, ampicillin, carbenicillin, cloxacillin, dicloxacillin, methicillin, nafcillin, oxacillin, phenethicillin, and ticarcillin; polypeptide antibiotics, such as bacitracin, colistimethate, colistin, polymyxin B; tetracyclines, such as chlortetracycline, demeclocycline, doxycycline, methacycline, minocycline, tetracycline, and oxytetracycline; and miscellaneous antibiotics such as chloramphenicol, clindamycin, cycloserine, lincomycin, rifampin, spectinomycin, vancomycin, and viomycin. Additional antibiotics' are described in “Remington's Pharmaceutical Sciences,” 16th Ed., (Mack Pub. Co., 1980), pp. 1121-1178.

Immunosuppression or immunosuppressive regimen, as used herein, refers to the treatment of an individual, for example a graft recipient with agents to diminish the immune responses of the host immune system against autoantigens or graft. Exemplary immunosuppression regimens are described in more detail herein.

Primary immunosuppressive agents include calcineurin inhibitors, which combine with binding proteins to inhibit calcineurin activity, and which include, for example, tacrolimus, cyclosporine A, etc. Levels of both cyclosporine and tacrolimus must be carefully monitored. Initially, levels can be kept in the range of 10-20 ng/mL, but, after 3 months, levels may be kept lower (5-10 ng/mL) to reduce the risk of nephrotoxicity. Adjuvant agents are usually combined with a calcineurin inhibitor and include steroids, azathioprine, mycophenolate mofetil, and sirolimus. Protocols of interest include a calcineurin inhibitor with mycophenolate mofetil. The use of adjuvant agents allows clinicians to achieve adequate immunosuppression while decreasing the dose and toxicity of individual agents.

Active ingredients in pharmaceutical compositions formulated for the treatment of various disorders are as described above. The active ingredient is present in a therapeutically effective amount, i.e., an amount sufficient when administered to substantially modulate the effect of the targeted protein or polypeptide to treat a disease or medical condition mediated thereby. The compositions can also include various other agents to enhance delivery and efficacy, e.g. to enhance delivery and stability of the active ingredients.

Thus, for example, the compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

The detection reagents, for example, one or more isotype-specific or glycosylation-specific reagents can be provided as part of a kit. Thus, the invention further provides kits for detecting the presence of pathogen-specific antibodies of interest in a biological sample. The kit may further comprise a diagnostic bait for detecting pathogen-specific antibodies of interest, which may include for example, a diagnostic pathogen or antigen array. Procedures using these kits can be performed by clinical laboratories, experimental laboratories, medical practitioners, or private individuals. The kits of the invention for detecting markers comprise genetically modified pathogen(s) and labeling reagents useful for evaluation. The kit can optionally provide additional components that are useful in the procedure, including, but not limited to, buffers, developing reagents, labels, reacting surfaces, means for detection, control samples, standards, instructions, and interpretive information.

In addition to the above components, the subject kits will further include instructions for practicing the subject methods. These instructions can be present in the subject kits in a variety of forms, one or more of which can be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that can be present is a website address which can be used via the internet to access the information at a removed site. Any convenient means can be present in the kits.

In some embodiments, the kit comprises a Borrelia burgdorferi diagnostic pathogen and one or more isotype-specific or glycosylation-specific reagents for detecting Borrelia burgdorferi pathogen-specific antibodies. In some embodiments, the kit comprises IgE-specific reagents for detecting Borrelia burgdorferi pathogen-specific IgE antibodies. In some embodiments, the kit further comprises one or more of an antihistamine, mast cell stabilizer, and an anti-IgE therapeutic agent.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

It is shown herein that comprehensive antibody isotype profiling identifies IgE as a key biomarker and instigator of immune pathology. Profiling immunoglobulin levels and ratios provide an indication of the presence of the bacteria, and assess the state of the immune response in each patient, which can be used to inform clinical decision making. A method for a comprehensive analysis of all antibody isotypes and their relative quantities in the serum is described in the Examples below.

Clinically, IgE is significant as it binds with high affinity to its Fc receptors on basophils and mast cells, triggering significant local histamine release, which further amplifies IgE-mediated immune responses while suppressing IgG-mediated immune responses. Basophil and mast cell degranulation upon antigen binding to IgE attached to the mast cell Fc receptor leads to histamine and other allergic factor release, causing extensive tissue damage, and can play a role in many of the signs and symptoms that have been reported, for example, in Lyme disease, including arthritis, malaise, fatigue, and cognitive impairment. Thus, the production of IgE can impact pathogenesis. The production of IgE and severity of the Type-2 immune response varies greatly among different strains of mice, suggesting that the diversity of symptoms reported by Lyme disease patients has to do with individual genetic makeup.

To investigate the individual immune responses in high resolution, a comprehensive serologic test was developed for the presence and relative quantities of antibodies of all antibody isotypes. The method utilizes anti-isotype and anti-subtype panel combined with the power and resolution of flow cytometry to measure binding of pathogen-specific antibodies from putative patients to live, GFP-expressing pathogen cells.

For example, whole, GFP-labeled bacteria are incubated with serum samples, and Bb-specific antibodies that bind to bacterial surface-epitopes are detected using a comprehensive secondary antibody panel analyzed by flow cytometry. This method allows profiling of the isotype repertoire of bound Bb-specific antibodies and determines a threshold for scoring positive for Bb infection. This method will have a lower rate of false positives because the substrate for the antibodies are whole bacteria and not a bacterial lysate that exposes conserved intracellular bacterial epitopes. Additionally, the resolution of flow cytometry provides very high sensitivity and reproducibility. With this high sensitivity, classical indicators of infection such as IgM and subtypes of IgG antibodies are detectable with in the first week of infection, whereas with the currently used methods the recommendation for reliable results is to test 8 weeks post infection. Strikingly, we also found IgE antibodies to Bb, an unanticipated response to a bacterial pathogen, both in animal models and in samples from two-tier positive patients analyzed at longer periods after infection.

For Borrelia infection, a susceptible mouse strain (C3H/HeJ) differs significantly in the kinetics and magnitude of their IgE response in comparison with a mouse strain that is resistant to Bb induced pathology. We also observe mast cell infiltrate into the swollen joints of Bb infected C3H/HeJ mice. Our data show that IgE-induced mast-cell degranulation is pathological in Lyme arthritis. As histamine release during mast cell degranulation is a major modifier of many pathways and could directly be responsible for how mast cell degranulation causes swelling around the joint, we tested the impact of anti-histamine treatment over the course of infection. Treatment with a histamine type 2 receptor antagonist (H2 blocker) administered in the drinking water of mice starting at 2 weeks post infection prevented ankle swelling, whereas type 1 histamine receptor antagonist had no effect. These findings indicate that mast cell degranulation release of histamine and signaling through type 2 histamine-receptors on target cells is responsible for the edema tibiotarsal swelling and can explain some clinical symptoms associated with Lyme disease that do not have a clear association to Bb infection.

The conditions for preparing Bb for use in the diagnostic are optimized; and fixation and preservation protocols developed to preserve bacterial epitopes and eliminate batch effects between runs. Fixation reduces biological safety concerns as it renders bacteria non-infectious. For example, iterations of aldehyde-based fixatives starting with formaldehyde and gluteraldehyde preparations at varying concentrations, immersion times, and temperatures which could best maintain surface epitopes without permeabilizing the outer membrane which would expose highly conserved intracellular proteins.

We currently use Bb at both exponential and stationary growth phases and test all samples in parallel with both phases. All samples from a given cohort are compared together after binding to the same batch of bacterial culture, same staining conditions, and same day flow cytometry analysis. This prevents batch effects within a cohort, but to compare results from one cohort to another we normalize bacteria numbers and concentration in the assay and compare percentage binding in comparison to bacteria that were incubated with secondary antibodies without prior incubation with serum.

Bb may be further genetically modified to increases specificity. Currently the Bb bait are genetically modified to express GFP for ease of positively identifying them by flow cytometry. The Bb can be further genetically modified to eliminate highly conserved surface proteins as desired. Alternatively the most immunogenic epitopes to which the antibodies bind are identified, and used to generate a panel of antigens as a substrate for screening.

Immunoglobulin heavy chains allow for distinction of 5 major isotypes of antibodies produced by the immune system. These are IgD, IgM, IgA, IgE and IgG, which can be further organized into subclasses (i.e. IgG1), each of which contain multiple isotypes that elicit distinct effector functions. Lyme diagnostics measuring bulk IgG or IgM not only fail to look for other major isotypes, but also miss the resolution of antibody subclasses which are optimal for detailed understanding of the patient's immune response. These assays use bacterial lysates which can denature key binding epitopes and, through display of intracellular proteins, may render results non-specific. These disadvantages confer low sensitivity, and render the test insufficient for detection of early infection. Consequently, the proportion of patients presenting to the clinic with an erythema migrans (EM) rash who are also seropositive using the CDC-recommended two-tier test is less than 30% in the critical early window when antibiotic therapy is most effective. In most cases, treatment with antibiotics such as doxycycline, amoxicillin, or cefuroxime is sufficient to resolve the infection if started in time. Therefore, it is critical to ascertain whether seronegative patients in early stages of Bb infection are true negatives by testing all of the antibody isotypes.

IgM and IgG anti-Bb antibodies identified by the current diagnostic methods provide a small window for identifying patients infected with Bb. With the inclusion of IgE and other antibody isotypes, we were able to improve the separation between two-tier validated Lyme patients and uninfected healthy controls. Even IgG1 alone by this method shows the sensitivity of the ELISAs that were performed, with a higher sensitivity than with western blots as seen in patients that had indeterminate values by western blot and clearly positive values by our flow cytometry-based assay. Adding in additional parameters, whether additional subtypes of IgG or other isotypes such as IgE further separated patients, where the ratios of IgE vs IgG subtype anti-Bb antibodies stratified healthy controls and patients into two distinct, unambiguous clusters. Additionally, we can identify patients that mount a non-canonical response to infection by identifying IgE in patients negative for IgG or IgM but positive in direct testing to avoid false negatives.

While IgG and IgM are considered the classic antibody isotypes that dominate the immune response to bacterial infection, IgA has also been implicated in neuroborreliosis. We will determine the level of all anti-Bb antibody isotypes and subtypes in pre and post-treatment patient samples. We will track whether levels of IgE or IgA, either alone or when compared to other antibody isotype levels, cluster patients who recover compared to those who do not post-treatment.

Approaches upstream of IgE which can eliminate the allergic response in Lyme Disease mice or patients are also utilized. Beyond antihistamine use or IgE blockade, longer lasting solutions are provided through immunomodulatory strategies further upstream of IgE binding of mast cells and histamine release. Approaches that act upstream of mast cell degranulation can reduce the IgE-mediated response to Lyme disease in mice and/or patients. Quilizumab is a monoclonal antibody that targets the M1-prime segment, CεmX, of membrane expressed IgE. This antibody crosslinks the membrane bound IgE antigen receptors on B cells, inducing IgE positive B cell apoptosis, decreasing the free, circulating IgE levels and inhibiting generation of IgE. To assess the efficacy of this anti-human antibody in-vivo, a genetically modified mouse model in which the human M1′ domain inserted into the mouse IgE locus rendering the IgE-producing B cells susceptible to depletion mediated by the clinically-approved antibody is used. These mice are back-crossed to C3H to assure sensitivity to Bb, infected with the pathogen and treated with Quizilumab antibodies. Combination therapy of Quilizumab with and without CD47 blocking reagents is tested to further improve IgE+B cell clearance and bacterial clearance.

A co-culture system is used to test the effectiveness of IgE+B cell depletion in B cells that have undergone IgE isotype switch upon Bb infection. In this same model an additional drug that can also deplete IgE+B cells is tested, XmAb7195 alone or in combination with CD47 blockade. XmAb7195 is the most comprehensive and interacts furthest upstream in the type II immune response, XmAb7195 is known for its reliable double mechanism. The mechanism sequesters free IgE in the serum to form complexes with FcγRIIß and IgE receptors on B cells, blocking IgE signaling. This inhibits IgE positive B cell differentiation, decreasing IgE-secreting plasma cells, as well as inhibiting the formation of IgE-positive B cells and decreasing free and total IgE. This mechanism does not affect antigen isotypes of other B cells. These drugs can decrease the response seen by circulating IgE and/or IgE positive B cells, decreasing the type II immune response. The higher upstream and more comprehensively we impede, the fewer free IgE and IgE positive B cells in the serum. This could decrease Lyme disease's effects and for a longer amount of time, relative to current treatments available.

### Example 1

Immunosorbent Assay with Diagnostic Pathogen

We have developed methods for using intact, genetically modified pathogens for optimal detection of pathogen specific antibodies. The methods utilize an immunosorbent assay linked with fluorescent, colorimetric or metallic detection assays, for simultaneous detection of multiple antibody isotypes, subtypes and glycosylation states. By examining multiple antibody isotypes/subtypes/glycosylation motifs in parallel, the ratios of these different antibody forms are determined, allowing for detailed sub-grouping of disease states, with a high-resolution indication of the immune response to the pathogen.

Genetically modifying the pathogens to fluoresce allows rapid identification of live, intact pathogens, which can be distinguished from dead pathogens and debris. Keeping the pathogens whole and exposing only surface proteins to pathogen specific antibodies also eliminates exposure of highly conserved intracellular proteins that compromise specificity.

Further genetic modification of the pathogens to eliminate highly conserved epitopes from being expressed on the pathogen surface generates a complex of maximum specificity for immunosorbent detection of pathogen specific antibodies, while maintaining proteins in their native confirmation for maximum antibody epitope recognition. These modified pathogens are also useful as vaccination strains for generation of highly protective antibodies.

Lyme disease, caused by the bacteria Borrelia burgdorferi, is an example of a disease which is notoriously difficult to accurately detect. Current diagnostic tests suffer either from lack of specificity, lack of sensitivity, or sensitivity only to one particular strain such that others test negative. The assays provided herein are shown to provide high specificity and sensitivity diagnosis, even in early stages of Borrelia infection. Furthermore, simultaneously resolving different antibody isotypes, subtypes, and glycosylation states allows stratification of patients into different subgroups within Lyme disease, which provides critical information for decision-making of therapeutic options.

This assay is shown to detect infection in live mice within the first week of infection from a small drop of blood. The assay also identified critical ratios between different types of antibodies that are informative for the state of the immune response. Whereas bacterial infections generally lead to the induction of protective IgG responses, we found that Borrelia infection in the laboratory mouse strain C3H also leads to induction of IgE responses, which are more typical of response to parasites or allergens. Combining the diagnostic information with imaging results and the other observations provides for advances in understanding of how different infection conditions trigger different immune responses.

### Results

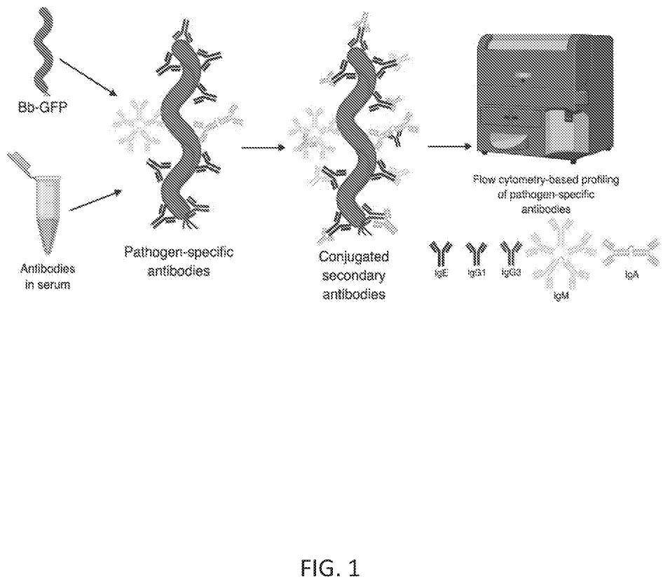

Shown in FIG. 1 is a schematic representation of the diagnostic immunosorbent assay. An intact Borrelia burgdorferi pathogen, free in suspension, is genetically modified to express green fluorescent protein (GFP). The pathogen is incubated with plasma from infected or uninfected hosts. After multiple washes, these pathogens, with any pathogen specific antibodies that are attached to the exposed surface proteins, are probed with a panel of secondary fluorescent-labeled antibodies that specifically bind to antibody isotypes, subtypes, and allotypes. This same strategy is applicable to antibodies conjugated to other molecules that can be used for detection, such as metals for mass cytometry. Antibodies specific to carbohydrate motifs can also be included in the panel.

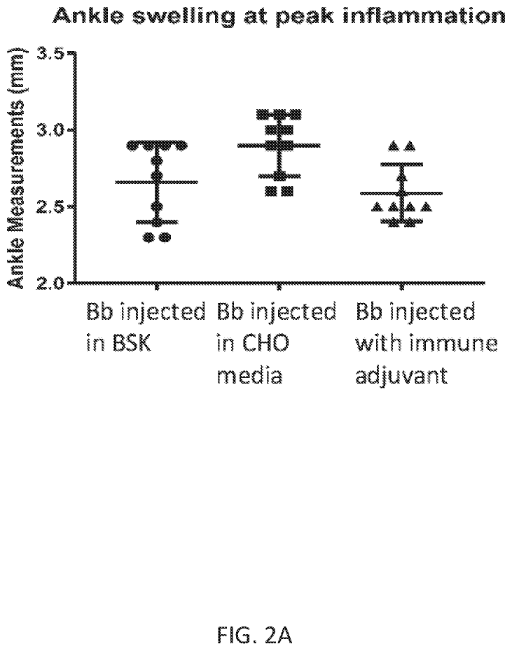

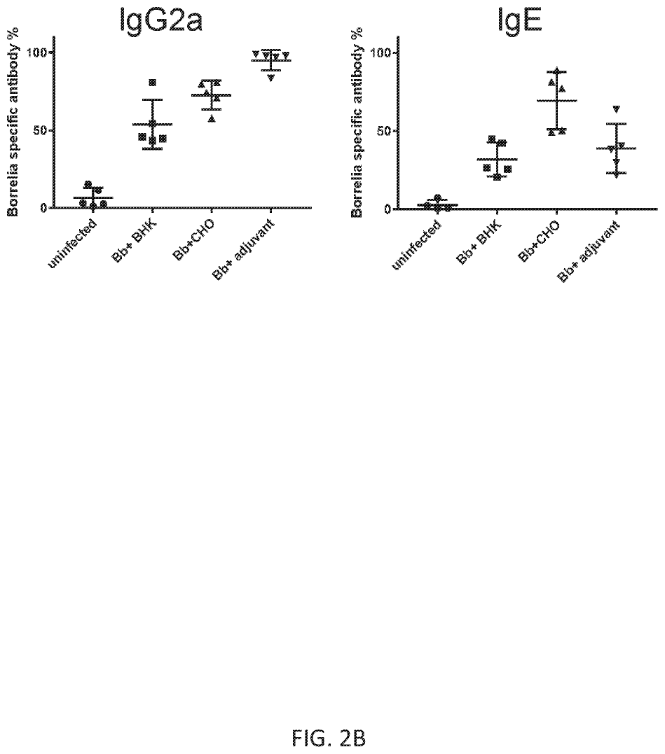

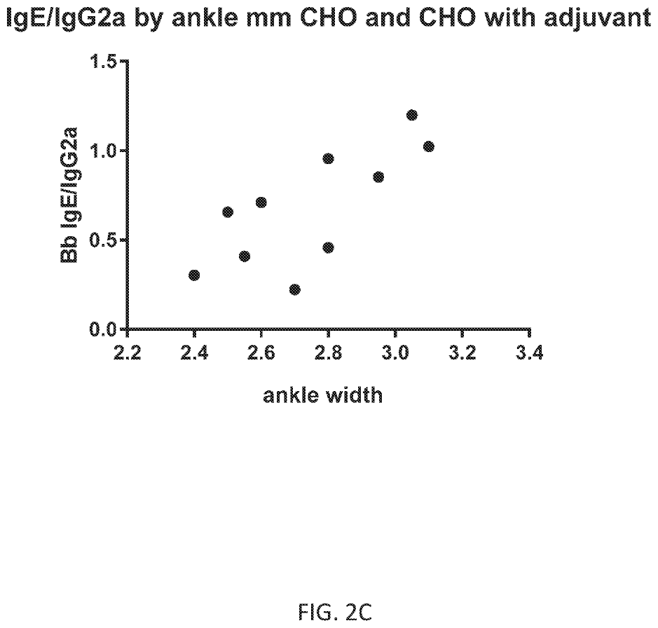

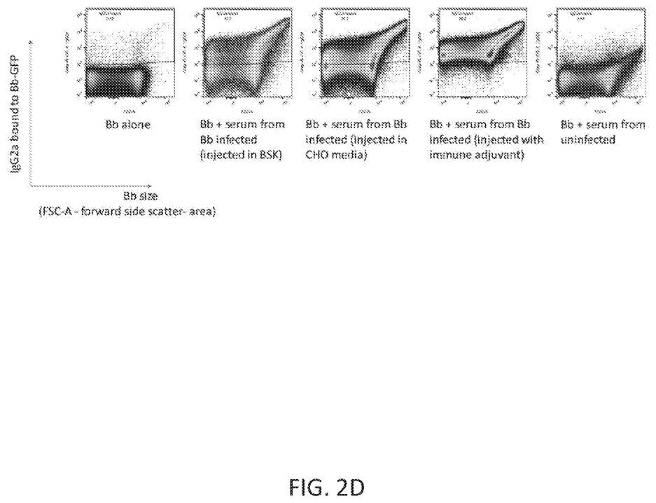

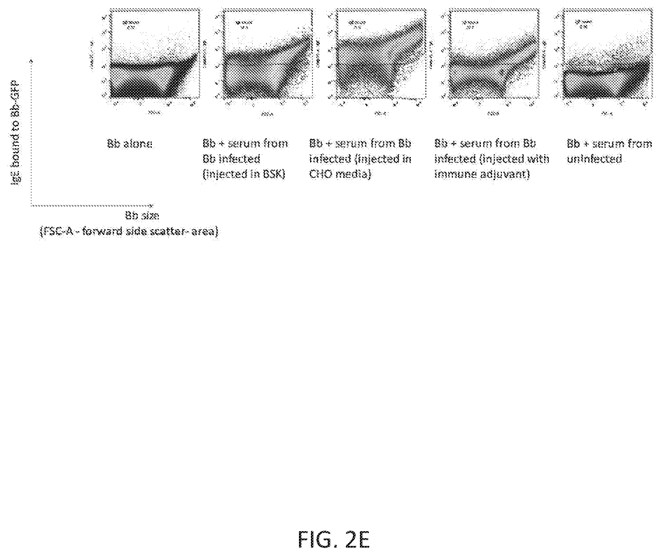

Shown in FIGS. 2A-2E are representative analyses of Borrelia-specific immune responses, and how they are impacted by infection conditions. Profiling multiple isotypes and subtypes allows for greater resolution of response to infection. C3H mice were infected with Borrelia burgdorferi-GFP and ankle swelling was measured over the course of a 67 day infection. Peak ankle swelling was observed at day 48 and antibody levels were measured at day 67 endpoint. FIG. 2A shows the ankle width (in mm) by group, where the highest degree of swelling and joint level inflammation was in the group that was infected with Borrelia injected in CHO media. This condition also induces clumping of the Borrelia into larger aggregates.

FIG. 2B show antibody isotype levels at day 67 post infection, depicted are IgG2a and IgE isotypes. Murine IgG2a is comparable to human IgG1, with the highest activating/inhibitory ratio of any of the IgG subtypes. IgE isotypes were also surprisingly found, as it is typically associated with parasitic infection and allergy responses.

Measuring a panel of borrelia specific antibodies by the methods disclosed herein allows improved resolution of the state of the immune response relative to conventional methods. This is important, as shown in FIG. 2C, where antibody ratios of IgE/IgG2a of both CHO media and CHO media with adjuvant conditions at day 67 post infection are correlated to peak ankle swelling measurements of day 48 post infection. FIG. 2D shows representative FACS plots of murine IgG2a antibody levels at day 67 post infection and FIG. 2E shows murine IgE at this time point.

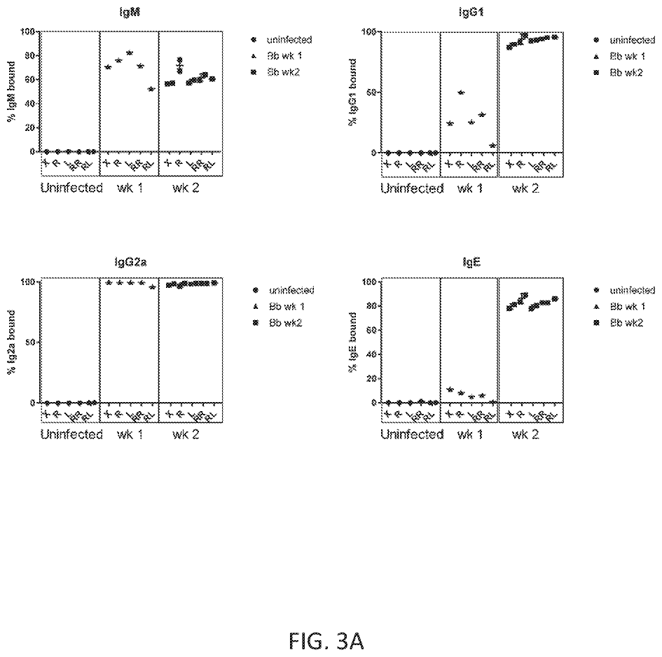

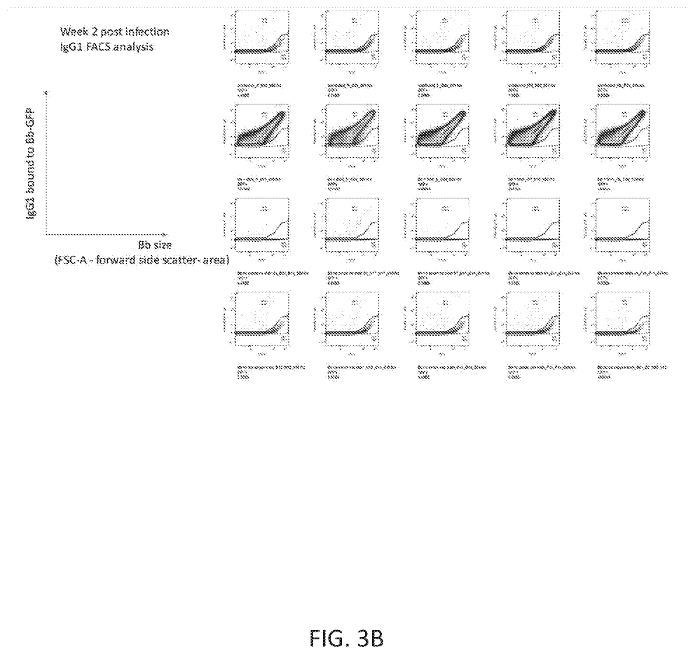

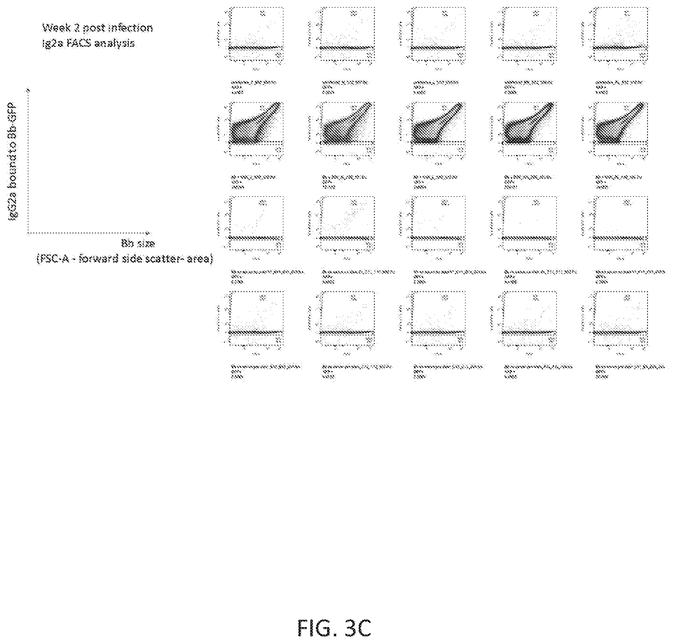

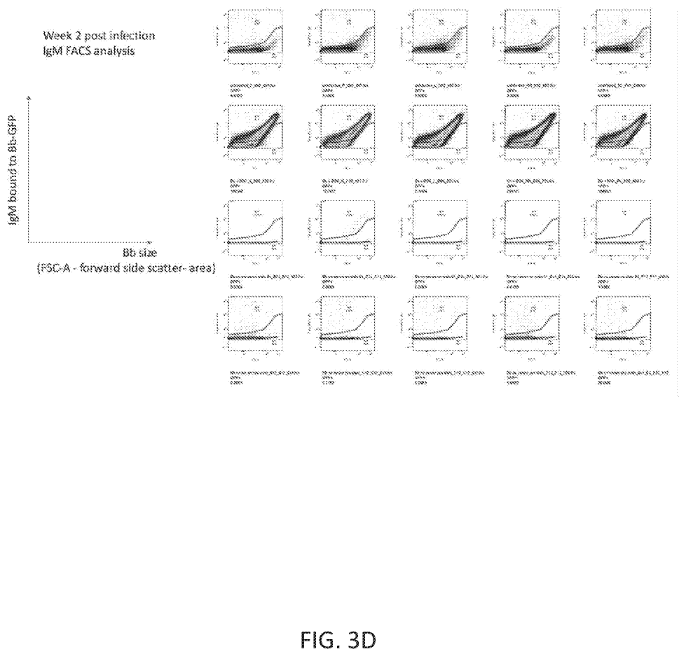

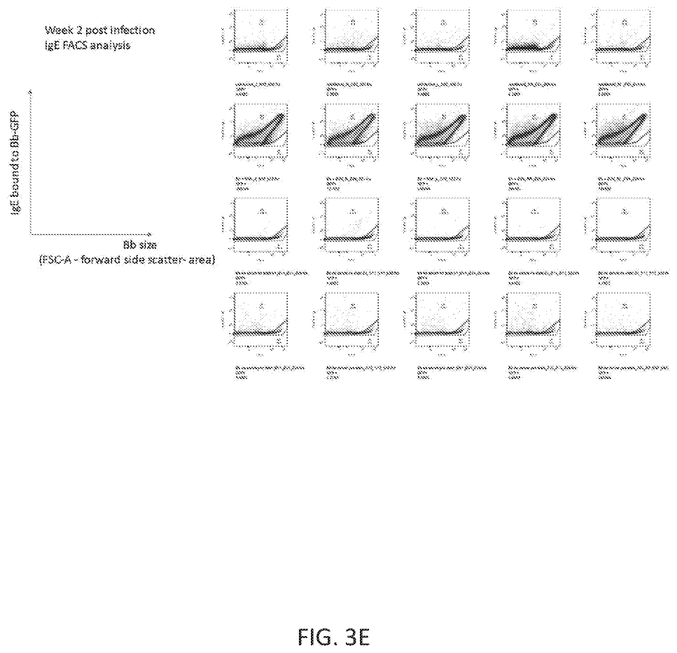

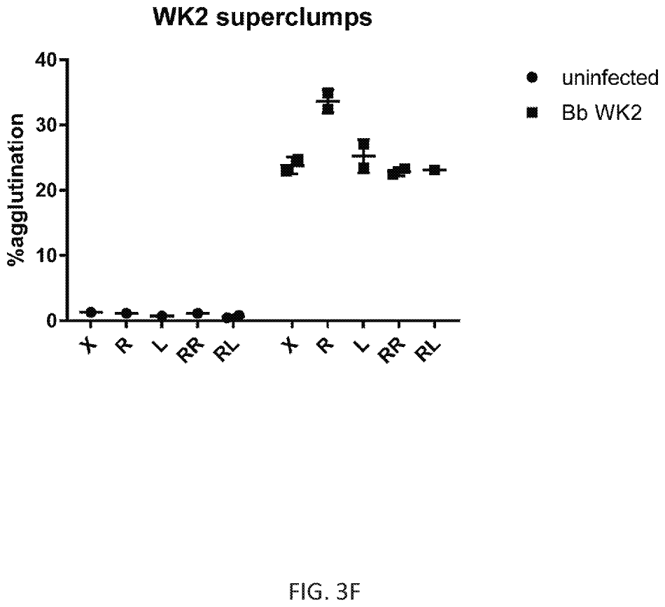

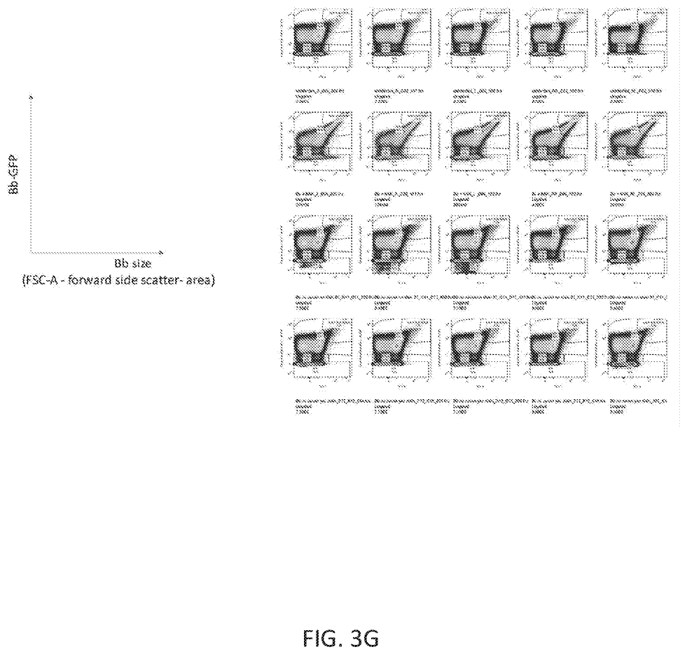

FIGS. 3A-3G provide time course analysis of antibody responses to infection, which are detectable 1 week post infection and are further increased at 2 weeks post infection, demonstrating a sensitive approach for early detection of infection. C3H mice were infected with Borrelia burgdorferi-GFP and plasma was isolated weekly, 10 μl of plasma was incubated overnight with 1×10⁶ Bb-GFP. After washing, the Bb-GFP bound to pathogen specific antibodies from the plasma was incubated with a panel of secondary mouse antibodies that were specific for IgM, IgG1, IgG2a and IgE.

FIG. 3A depicts murine antibody levels 1 week and 2 weeks post infection in comparison to uninfected mice. FIGS. 3b-3e are representative FAGS plots of murine IgG2a antibody levels at week 2 post infection for each of IgG1, IgG2a, IgM, IgE. The first row is plasma from uninfected mice. Second row contains plasma from infected mice. Third row contains Bb-GFP only, without plasma or secondary antibodies. The fourth row contains Bb-GFP and all secondary antibodies but no plasma. This last condition is the measure of noise, while the first condition of uninfected plasma is used to set the gates for each antibody of a % of the bound antibody. FIG. 3F measures murine serum induced bacterial agglutination as assessed by FAGS analysis 2 weeks post infection in comparison to uninfected mice. FIG. 3G is a representative FAGS analysis of the size of the Bb-GFP.

### Example 2

Immunosorbent Assay with Borrelia-GFP Diagnostic Pathogen

The diagnostic approach of immune state of response to Borrelia infection is adapted for detecting early and late infection in humans, and to systematically increase the specificity of the diagnostic to near 100%.

Secondary panels specific for human antibody isotypes, subtypes and allotypes are tested on healthy individuals to determine background levels of binding to Borrelia-GFP. Borrelia used in the immunosorbent assay are further genetically modified to increase specificity. Flagella are highly conserved between different bacterial species. Flagella mutants are analyzed in comparison to Bb-GFP in patient plasma samples from sequential time points over the course of infection in comparison to Borrelia naïve controls. Surface proteins exposed on Bb-GFP are examined for their level of conservation with other bacterial species and systematically mutated by order of conservation for further increases in specificity. Clinical isolates are collected, and GFP is genetically introduced. Flagella and other genes as necessary are genetically modified to ensure that this detection approach is not geographically restricted to one form of Borrelia, but can detect all Lyme disease causing strains.

Sensitivity of the assay is optimized by changing plasma incubation times with bacteria, secondary antibody concentrations and voltages by flow cytometry or changing antibody conjugates and the detection method.

Human patient samples are collected with treatment history to utilize the diagnostic approach to subset Lyme disease symptoms into specific subgroups defined by the state of the immune response to the Borrelia infection.

Patient plasma samples are collected from healthy controls and compared to samples from individuals who are positive for Borrelia antibodies using current serology approaches separated by those whose symptoms were relieved with antibiotic treatment and those with persistent symptoms. Antibody subtype ratios are examined to identify indications for distinguishing patients who have or have not be responded to treatment.

Patient plasma samples are collected at the time of presentation with erythema migrans (early infection with Borrelia) and the diagnostic strategy is compared to current serology diagnostics weekly for the course of 8 weeks. Patients are followed for response to treatment. Samples are analyzed for indicators of infection through our diagnostic strategy that are apparent prior to current serology approaches. IgE is examined in patients who in early weeks test negative with current serology which only probes IgM and bulk IgG.

The diagnostic strategy for detection of immune state is expanded to response to other pathogens. The fungal infection Aspergillus can cause very severe problems when lung damage is present, but to identify infection with aspergillus, it must be extracted from the respiratory tract and cultured. Samples of genetically modified aspergillus, containing different fluorescent molecules and in different forms are tested with patient serum of patients with active infection and the human secondary antibody panel to characterize antibody responses to aspergillus infection and to assess if the diagnostic strategy is applicable in fungal infections.

Infection with the intracellular parasite Toxoplasma gondii is very prevalent, but severely undertested. As Toxoplasma gondii is very amenable to genetic modification and many genetic mutants are already available, the diagnostic strategy is tested in toxoplasma naïve healthy controls in comparison to individuals with documented toxoplasma exposure and active toxoplasma infection.

A high-resolution analysis at the state of the immune response to a pathogen allows a better mechanistic understanding of different symptoms as they correlate with the effector functions triggered by the different antibody heavy chains and glycosylation motifs. Genetically modifying intact organisms to eliminate highly conserved surface exposed proteins allows enhanced specificity for antibody detection by keeping pathogen proteins in their native confirmation but retaining only the surface proteins that are highly specific to a given pathogen.

The ability to simultaneously assess different antibody subclasses that are currently overlooked during infection may improve detection and treatment of disease. The ratios between antibody types that are bound on highly specific model pathogens used as the bait in this immunosorbent diagnostic strategy may allow for a high sensitivity approach with customizable specificity.

### Materials and Methods

Genetic Modification of Borrelia. Performed as described by Moriarty et al. (2008) PLOS Pathog. 4(6):e1000090. Briefly, for construction of GFP expression plasmid pTM61, the terminator sequences (T1×4), rbs, B. burgdorferi flaB promoter and GFP coding sequences from pCE320(gfp)-PflaB were PCR-amplified with flanking SacI and KpnI sites, using primers B696 (5′-ccggagctcatgataagctgtcaaacatgag-3′) and B697 (5′-ccggtacctcagatctatttgtatagttcatc-3′), and cloned into pCR Blunt II-TOPO (Invitrogen Canada, Burlington, ON) with the insert SacI site proximal to the vector PstI site, to make plasmid pTM41. This insert could not be cloned into the gentamycin-resistant version of the pBSV2 shuttle vector (pBSV2G), presumably because replication origins and copy number sometimes affect the expression and toxicity of fluorescent proteins in E. coli. Therefore, a modified shuttle vector, pTM49, was constructed, in which the colEl ori of pBSV2G was removed by restriction digestion with enzymes MluI and SnaBI, and replaced with an MluI/SnaBI fragment from pCR Blunt II-TOPO containing the pUC ori. The (T1×4)-PflaB-gfp cassette from pTM41 was cloned into the SacI/KpnI sites of pTM49 to generate pTM61.

All strains were grown in BSK-II medium prepared in-house. Electrocompetent infectious B. burgdorferi strain B31 5A4 NP1 and non-infectious strain B31-A (both B31-derived) were prepared as previously described. Liquid plating transformations were performed with 50 μg pTM61 in the presence of 100 μg/ml gentamycin. Gentamycin-resistant B. burgdorferi clones were screened for: 1) the presence of aacC1 sequences by colony screening PCR performed with primers B348 and B349 as described; and 2) GFP expression by conventional epifluorescence microscopy. The presence of the pTM61 plasmid in non-integrated form in fluorescent strains was confirmed by agarose gel electrophoresis of total genomic DNA prepared on a small scale. PCR screening for native plasmid content was performed and indicated that one fluorescent infectious B. burgdorferi clone (GCB726) contained all endogenous plasmids except cp9, which was displaced by the cp9-based pTM61 construct. Non-infectious strain GCB705 was used for experiments with non-infectious B. burgdorferi. PCR screening for native plasmid content indicated that GCB705 contained the same plasmids as the B31-A parent. Plasmids Ip25, Ip28-1 and Ip36 are known to be essential for infectivity.

Deletion of flagella. Deletion is performed as described in Lin et al. (2015) mBio 6(3): e00579-15. B. burgdorferi B31 derivative 5A18NP1 is an infectious, moderately transformable clone used for generation of fliH and flil mutants. 5A18NP1 is a genetically engineered clone in which plasmids Ip28-4 and Ip56 are missing and bbe02, encoding a putative restriction-modification enzyme, has been disrupted. A fliH mutant is obtained by random, signature-tagged transposon mutagenesis using the Filmed-based suicide vectors pGKT-STM5 or pGKT-STM10. The B. burgdorferi strains are grown in Barbour-Stoenner-Kelly II (BSKII) medium supplemented with 6% (vol/vol) rabbit serum and appropriate antibiotics or on semisolid agar plates at 34° C. in 3% CO₂. 5A18NP1 is cultured in medium containing 200 μg/ml kanamycin, and the transposon mutants are cultured in the presence of both kanamycin and 40 μg/ml gentamicin. Additionally, streptomycin (50 μg/ml) is included in complemented transposon mutant cultures. The plasmid content of each clone can be determined using a Luminex-based procedure.

Complementation shuttle vectors are constructed by inserting constitutive expression constructs pflaB::fliH, pflaB::flil, and pflaB::fliHI into the shuttle vector pKFSS1. Briefly, the flaB promoter (PflaB), the genes fliH and flil, and the contiguous fliHI gene cluster are amplified by PCR using primers with engineered restriction sites and cloned into the PCR2.1 vector (Life Technologies, Grand Island, N.Y.). PflaB is first subcloned into pKFSS1 at the SacI and KpnI sites, and then the fliH, flil, and fliHI genes are fused to the 3′ end of flaB promoter at the KpnI site and PstI site, resulting in complementation plasmids. The resultant constructs are confirmed by PCR, restriction patterns, and sequencing of FOR products. A fliH mutant and flil mutant are transcomplemented by transforming with the complementary shuttle vectors. Electroporation of B. burgdorferi is performed as described previously. Transformants are confirmed by FOR and sequencing.

The morphology, motility, and swimming ability of fliH and flil mutants, complemented clones, and the parental strain are determined in BSKII medium in the presence and absence of 1% methylcellulose under dark-field microscopy examination.

Staining serum for diagnostic analysis. Antibodies are purchased from Biolegend. PE anti-mouse IgE, Clone RME-1, Catalog no. 406908; AlexaFluor 647 anti-mouse IgM, Clone RMM-1, Catalog no. 406526; PE/Cy7 anti-mouse IgG2a, Clone RMG2a-62, Catalog no. 407114; APC/Cy7 anti-mouse IgG1, Clone RMG1-1, Catalog no. 406620.

The blood sample is prepared by collection. Soft spin at 400 RCF for 5 minutes at 4 C. Remove plasma into clean tubes. Hard spin at 10,000 ROE for 10 minutes at 4 C. Remove 10 μL plasma into well plate, using same scheme from each week. Make duplicates if possible. Shake and pour Bb into a clean reservoir. Using multichannel pipette, pipette 150 μL into two rightmost columns in V-bottom plate. Spin for 10 minutes at 1500 RCF/g at 4 C.

Staining is performed with 50 μl antibody solution/sample, diluted into 2 ml of PBS, the 50 μl/well of serum sample. Stain on ice for 18 minutes in the dark. Add 150 μl. PBS, spin for 6 minutes at 1500 RCF/g at 4 C. Pipette off supernatant, wash twice with 200 μl. PBS, and spin for 6 minutes at 1500 RCF/g at 40.

For flow cytometry, fix sample in 4% PFA for 10 minutes in the dark at room temperature. If analyzing immediately, run in PFA, if keeping cells for more than 24 hours, wash with 200 μl PBS

### Example 3

### Borrelia-Specific IgE Antibodies Indicate a Subset of Lyme Disease Cases that Will Benefit Therapeutically from Intervention of IgE Mediated Pathology

We have developed a diagnostic test using a comprehensive anti-isotype panel allowing the detection of all antibody subtypes on intact Bb by flow cytometry, as described in Examples 1 and 2. Whole, bacteria are incubated with serum containing Bb-specific antibodies, which then bind to bacterial surface epitopes. After washing, we use a comprehensive secondary antibody panel, analyzed by flow cytometry, to profile the isotype repertoire of bound Bb-specific antibodies and determine a threshold for scoring positive for Bb infection (FIG. 1). Classical indicators of infection, such as IgM and subtypes of IgG antibodies, are detectable with our diagnostic method as early as day 4 of infection.

Strikingly, we found IgE antibodies to Bb both in animal models and in samples from CDC-positive patients analyzed at longer periods after infection, an unanticipated response to a bacterial pathogen (Table 1). Bb-specific IgE antibodies have been previously documented sporadically in Lyme disease patients, but, despite this, current diagnostic strategies do not examine IgE. With current diagnostics, individuals who produce Bb-specific IgE antibodies to the exclusion of other isotypes may be classified as negative for Lyme altogether^{2,4,5}.

Mast cells bind IgE, triggering significant local histamine release, which further amplifies IgE-mediated immune responses while suppressing IgG-mediated immune responses⁶. Mast cell degranulation upon antigen binding to the mast cell IgE receptor leads to histamine and other allergic factor release, causing extensive tissue damage, and may play a role in many of the signs and symptoms that have been reported in Lyme disease, including arthritis, malaise, fatigue, and cognitive impairment^{6,7}. Thus, the production of IgE in response to Bb impacts the pathogenesis and affirms the need for more comprehensive testing.

Current testing identifies anti-Bb IgM and IgG antibodies only, and we propose that individuals who produce more IgE antibodies to Bb than IgG because of genetic susceptibility and immune skewing will test negative with current methods. Consequently, they may not receive treatment or resolve their infection, both because treatment is contraindicated for them based on their test results, and because they mount a Type-2, not a Type-1, response. Thus, the development of new and comprehensive diagnostics is imperative. Furthermore, the production of IgE to Bb may explain some clinical symptoms associated with Lyme disease that do not have a clear association to Bb infection, such as joint swelling, fatigue, malaise, cognitive impairment and more.

We are currently screening patients' sera to determine how prevalent IgE production is following Bb infection and investigating the following questions: 1) Is the Type 2, IgE-mediated immune response causative of Lyme disease symptoms that are associated with Bb infection, and is the pathology mediated by a particular cell type or molecule (e.g. histamine)? and 2) Can detection of anti-Bb IgE antibodies in the serum of patients be utilized for improving diagnostics and informing therapeutic interventions?

Importantly, the production of IgE and severity of the Type-2 immune response varies greatly between different strains of mice, suggesting that the diversity of symptoms reported by various Lyme disease patients has to do with the genetic makeup of each individual contracting the disease. Allergic predilections have long been observed to be inherited in families. If our hypothesis is correct, we might be able to better understand why the clinical presentation of Lyme disease symptoms varies so greatly between patients and why some patients continue to suffer from these symptoms after antibiotic therapy. Current genome-wide association studies of particular alleles and allergic disorders could give clues as to how to classify Bb-infected patients whose Lyme disease symptoms did or did not resolve.

TABLE 1
A discovery panel of Lyme disease patients and controls demonstrates the concordance of our novel diagnostic
strategy with existing methods and demonstrates our test’s ability to provide more detailed information.

| | Stony | | C-6 | | | | | | |
|---|---|---|---|---|---|---|---|---|---|
| | Brook | | Peptide | | | | | | Two- |
| | ELISA | | ELISA | | Western | | Western | | Tier |
| | Result | | Result | | Blot IgM | | Blot IgG | | Result |
| ID | 1 | 2 | 1 | 2 | 1 | 2 | 1 | 2 | 1 |
| 515 | NEG | | NEG | | NEG | | IT | | NEG |
| 526 | NEG | | NEG | | IT | | NEG | | NEG |
| 538 | NEG | | NEG | | IT | | IT | | NEG |
| 610 | NEG | | NEG | | IT | | NEG | | NEG |
| 611 | NEG | | NEG | | IT | | IT | | NEG |
| 674 | NEG | | NEG | | IT | | IT | | NEG |
| 585 | POS | | POS | | POS | | IT | | POS |
| 640 | POS | POS | POS | POS | NEG | IT | POS | POS | POS |
| 663 | IT | POS | POS | POS | POS | POS | IT | IT | POS |
| 673 | POS | POS | POS | POS | POS | POS | POS | IT | POS |
| 677 | POS | | POS | | IT | | POS | | POS |
| | Two- | | | | | | | | |
| | Tier | | | | | | | | |
| | Result | IgG1 | | IgM | | IgG3 | | IgE | |
| ID | 2 | 1 | 2 | 1 | 2 | 1 | 2 | 1 | 2 |
| 515 | | 0.34% | | 1.88% | | 0.24% | | 1.08% | |
| 526 | | 0.50% | | 0.32% | | 0.68% | | 0.43% | |
| 538 | | 0.18% | | 0.25% | | 0.23% | | 0.07% | |
| 610 | | 0.41% | | 0.063% | | 0.36% | | 0.20% | |
| 611 | | 2.58% | | 0.07% | | 1.26% | | 0.13% | |
| 674 | | 4.28% | | 3.84% | | 3.53% | | 1.25% | |
| 585 | | 26.0% | | 2.56% | | 15.0% | | 3.88% | |
| 640 | POS | 18.1% | 18.0% | 0.32% | 0.51% | 14.0% | 14.7% | 3.19% | 5.96% |
| 663 | POS | 1.47% | 18.6% | 3.29% | 18.10% | 0.79% | 1.76% | 0.37% | 14.60% |
| 673 | POS | 45.6% | 24.4% | 40.6% | 41.6% | 23.6% | 14.7% | 7.32% | 4.54% |
| 677 | | 16.1% | | 0.064% | | 11.9% | | 3.78% | |

Samples provided by the Lyme Disease Biobank of patients and endemic healthy controls from Long Island, N.Y. collected in 2017. Patients 640, 663 and 673 had their blood drawn at two time points, and serial results are shown as 1 and 2, respectively. These 3 patients were treated with Doxycycline between visits. The other patients only had one blood draw. Patients 611 and 674 were diagnosed as negative for Lyme using the two-tier method following an indeterminate WB for both IgM and IgG, but have detectable IgG and IgM levels higher than other confirmed negatives. Infected patients were all found to make IgE antibodies to Bb, though absolute values varied considerably between individuals. The Stony Brook ELISA uses whole-cell lysate of B31 Bb, while the C-6 Peptide ELISA uses only the 0-6 component from Bb. NEG=negative; POS=positive; IT=indeterminate.

### Example 4

### Immune-Modulatory Strategies Directed at Clearance of Bb Infection and Alleviation of Immune Reaction-Based Pathology

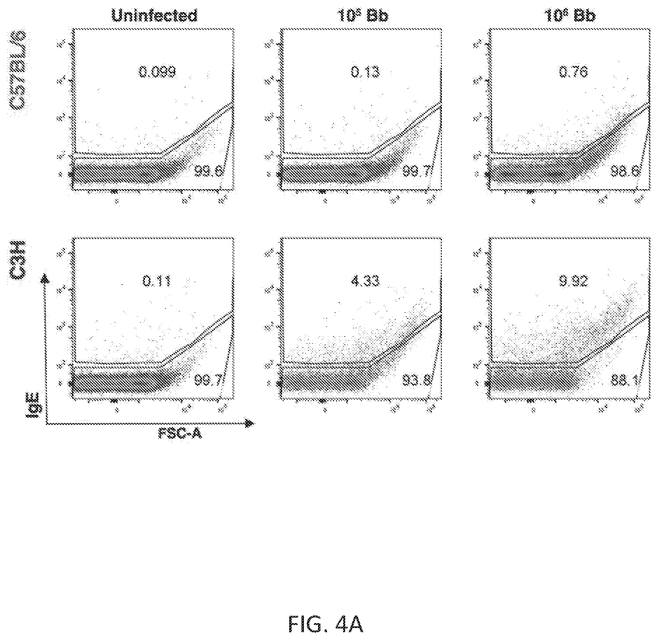

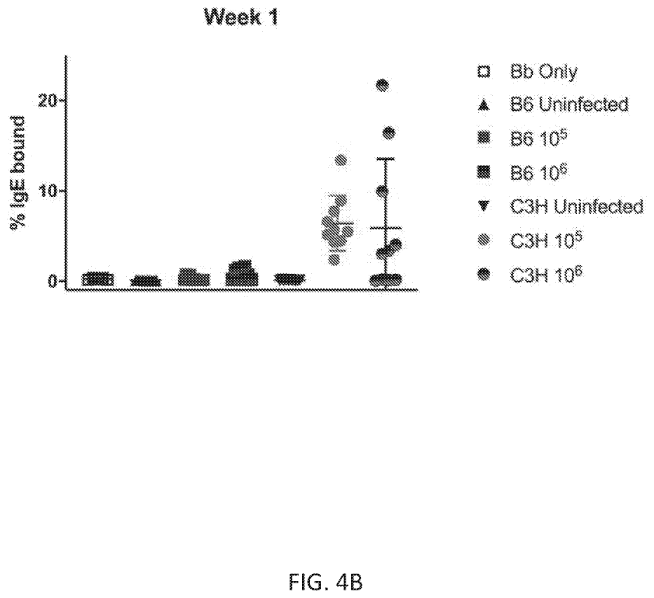

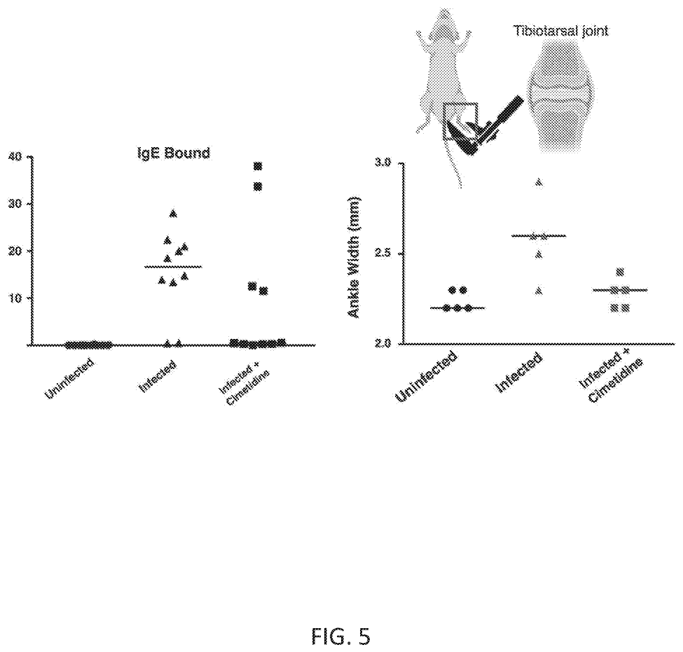

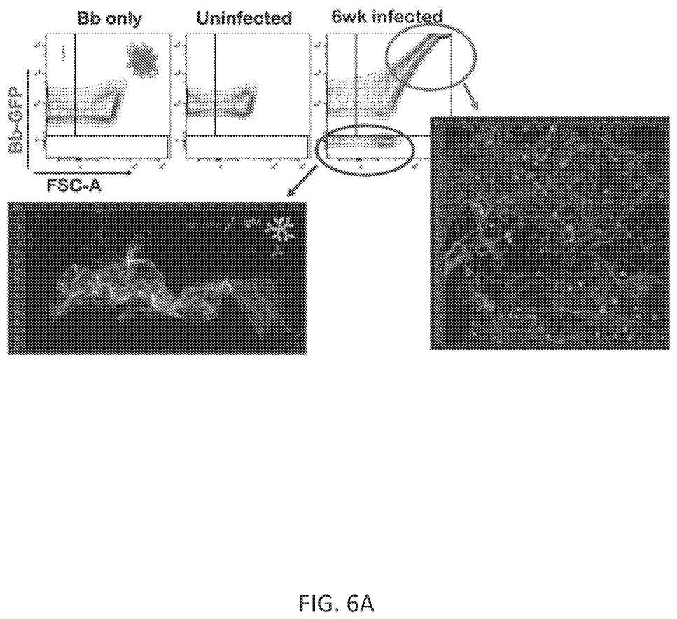

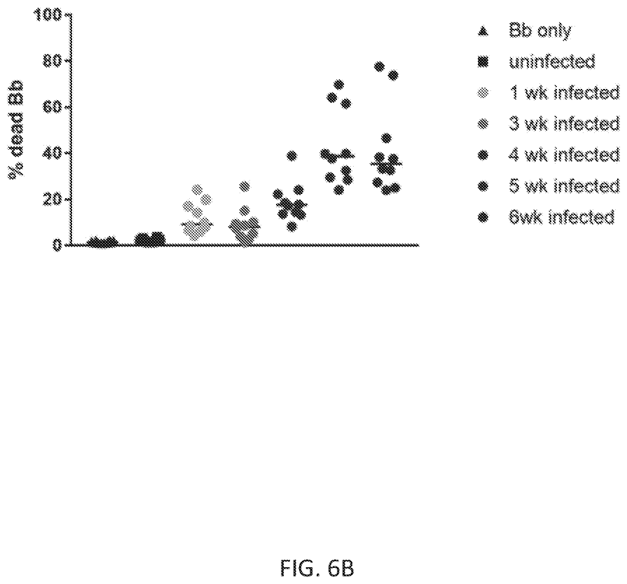

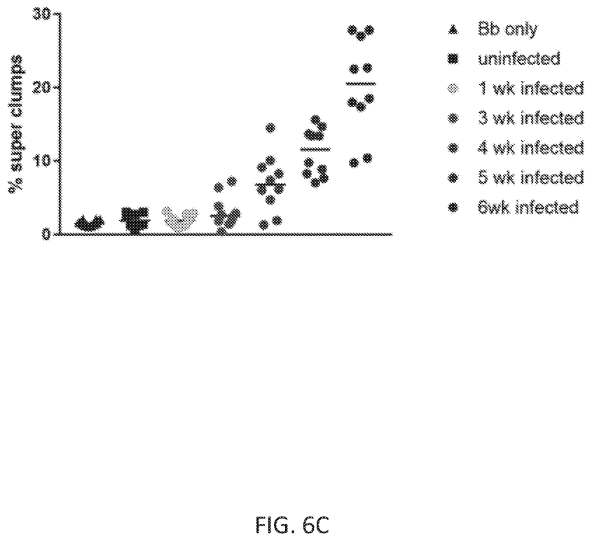

Our diagnostic strategy identified infection conditions that induce significant levels of Bb-specific IgE (FIGS. 3 and 4). IgE antibodies are a hallmark of Type-2 immune responses, along with recruitment of mast cells, basophils, eosinophils and histamine release. This was an unexpected response to a bacterial pathogen, and we became interested in its relevance to Bb pathogenesis. The implications of Type-2 immune responses to Bb and therapeutic immunomodulation in combination with standard antibiotic therapy is investigate. Insofar as Lyme disease is difficult to treat and causes much morbidity, anti-allergic compounds that currently exist that might ameliorate the disease are identified.

The generation of IgE vs other immunoglobulin isotype antibodies in vivo involves a number of factors, beginning with the activation of ‘helper’ T cells that secrete cytokines IL-4 and IL-13, which bind to antigen-activated B cells and trigger the immunoglobulin class switch toward IgE. Further activation by antigen leads to the formation of plasma B cells that secrete IgE. Secreted IgE binds to mast cells and basophils, cells with high-affinity receptors for the Fc portion of IgE (FcεRI) and forms long-lasting cross-links between additional receptor-bound IgE antibodies. The interaction between allergen, IgE, and FcεRI+ cells leads those cells to degranulate, releasing histamine and other molecules. Most of the inflammation, swelling and pain of these allergic responses comes from the degranulation products. This pathway provides a variety of targets to consider, and each step can be examined for effective therapeutic intervention. Because many patients seek relief from symptoms that can result from an allergic-type immune response, and because many antihistamines are affordable and widely available, making them relatively simple to incorporate into a treatment plan, the potential of antihistamine treatment in Bb infection is investigated.

Histamine has four unique receptors and impacts many non-immunological aspects of physiology. We are elucidating the nature of histamine's role in Lyme disease symptoms by infecting mice with Bb, confirming IgE production by diagnostic immunoassay and treating them with various antihistamines to see if their arthritic swelling is resolved. By using different histamine receptor antagonists (H1: diphenhydramine, loratadine, H2: cimetidine, ranitidine, H1&2: doxepin, H3: thioperamide, clobenpropit), it is determined which histamine-related pathologies can be inhibited.

If antihistamines alone are insufficient, approaches that act upstream to mast cell degranulation and reduce the IgE-mediated response to Lyme disease in mice and/or patients are used. Mast cells are depleted in a mouse model to determine if this approach can eliminate any of the allergy. An immunotherapeutic combination that eliminates mast cells for in vivo depletion is anti-c-kit antibody, alone or in combination with anti-CD47.

Additionally, the efficacy of anti-IgE antibodies is tested, which may a) lower the tissue level of IgE, b) eliminate both IgE and mast cells, and c) eliminate IgE+ memory B cells. These studies illuminate the role of the Type-2 immune response to Bb, examining the impact of IgE and histamine on Lyme disease pathogenesis and symptomatic presentation.

### Example 5

### Immune Profiling Platform to Rapidly Stratify Lyme Disease Patient Subsets by the Type of their Immune Response and Hallmarks of their Clinical Presentation

As described in Examples 1 and 2, we have developed a method for a comprehensive analysis of all Bb-specific antibodies, their isotypes, and their relative quantities in the serum. The method utilizes the power and resolution of flow cytometry to measure binding of Bb-specific antibodies found in the serum to live, GFP-expressing individual Bb bacteria, as demonstrated in FIGS. 1-4. Furthermore, this technique measures the impact of the immune response on live bacteria; it allows for quantification of Bb-immune complexes and levels of bactericidal antibodies. Using this technique, we have identified unexpected antibody responses to Bb which is not tested by current diagnostic methods and may therefore contribute to false negatives in current diagnostics that focus solely on IgM or IgG responses.

We are applying this method to the systematic characterization of the distribution of anti-Bb antibodies of various isotypes in the serum of individual patients, and then correlating the clinical symptoms of each patient with specific patterns of their humoral immune responses. To this end, human patient samples and treatment history are collected and the diagnostic approach utilized to classify patients presenting with the broad umbrella of Lyme disease symptoms into specific subgroups.

Serum samples are collected from healthy controls and compared to samples from individuals who were positive for Bb antibodies using current serology approaches. Confirmed patients are further categorized by those whose symptoms were resolved with antibiotic treatment and those with persistent symptoms. Antibody subtype ratios are examined to identify indications for distinguishing patients who have or have not responded to treatment. Additionally, patient serum samples are collected at the time of presentation with the characteristic erythema migrans rash to assess performance of our diagnostic strategy in early infection. Our diagnostic is compared weekly to current serology diagnostics over the course of 8 weeks. Patients are also followed for their response to treatment. To compare the sensitivity of our new method to the existing diagnostics, samples are analyzed for indicators of infection that appear early in infection prior to their appearance using traditional serology approaches. IgE are specifically examined in patients who test negative with current serology in the early weeks of their infection.

### Example 6

### Immune Profiling after Malaria Infection

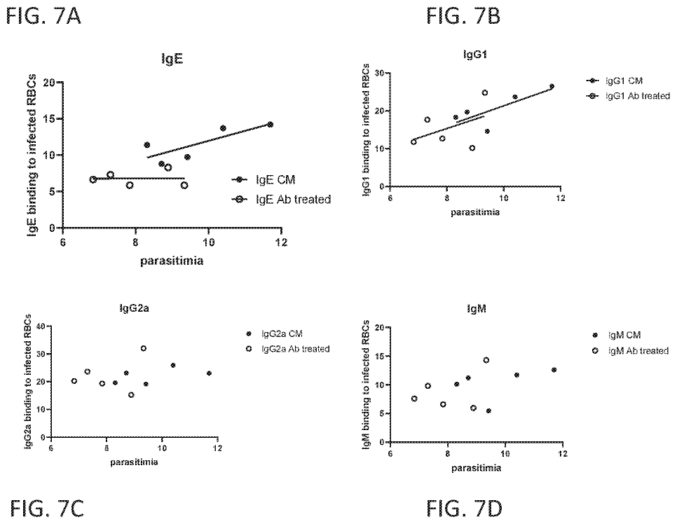

To test the ability of this immune profiling method to detect antibodies as they bind to infected red blood cells vs non-infected red blood cells, serum was used from mice which develop experimental cerebral malaria (ECM) in the Plasmodium berghei ANKA (Pb-A) murine model. In this murine model of ECM, 60-100% of C57BL/6 mice will develop symptoms that resemble the clinical features of human CM during the cerebral phase (days 6-10) of infection. Mice that do not develop ECM will instead develop severe anemia and hyper-parasitemia after day 15 post-infection that is also lethal. C57BL/6 mice infected with 106 Pb-A parasites were treated with either PBS or antibodies on day 3 post-infection and serum antibodies from those mice were assessed for binding to infected vs uninfected red blood cells from one day 6 infected mouse were tested. 20 ul serum was incubated with 5 ul of whole blood, cells were incubated for either 30 minutes or overnight and then stained with secondary antibodies against the antibody types and analyzed by flow cytometry. Binding of each antibody type to uninfected red blood cells was minimal, and binding of each antibody type to infected red blood cells which were GFP positive is shown. Data is shown in FIG. 7A-7D.

### Example 6

### Immune Profiling after Aspergillus Infection

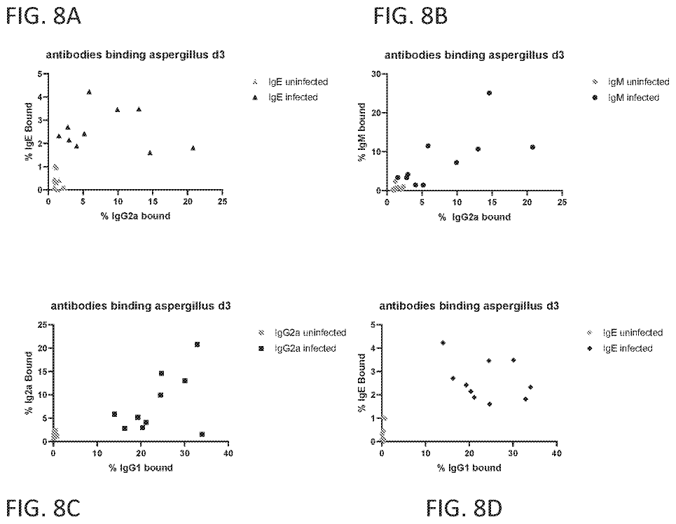

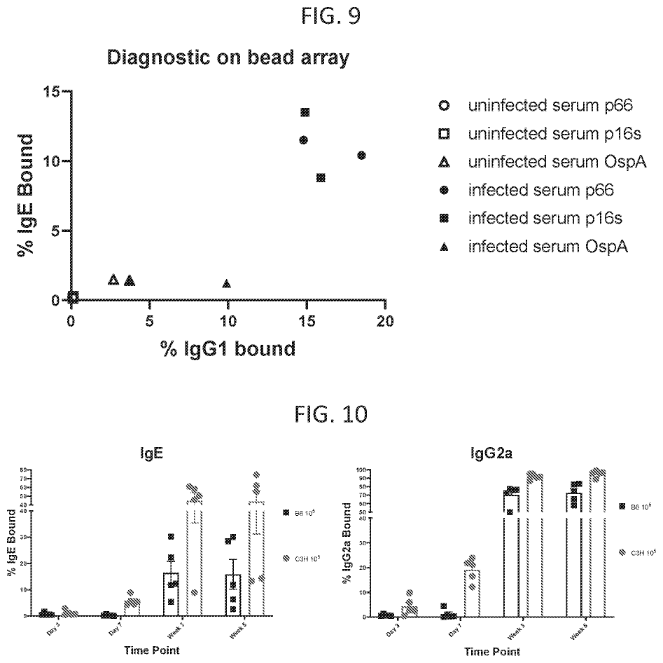

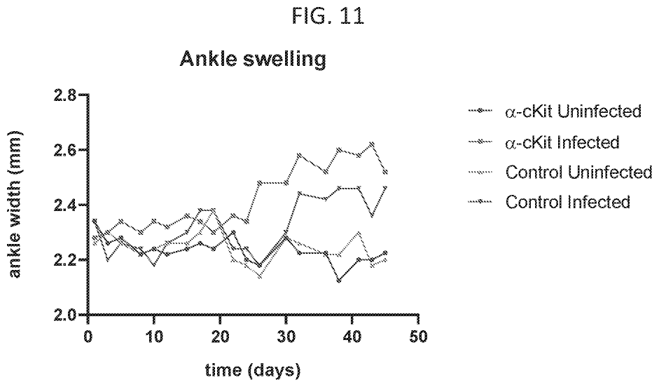

Serum from mice day 3 post infection with Aspergillus fumigatus was incubated with conidia of Aspergillus fumigatus either overnight or for 1 hr and then stained with antibodies against mouse IgG1, IgE, IgM and IgG2a. While IgG2a vs IgM did not show much separation between infected and uninfected groups, IgE vs IgG2a showed separation. Better separation was seen with IgG2a vs IgG1 and the best separation between groups clearly distinguishing positive and negative samples was IgE vs IgG1. Data shown in FIG. 8A-8D.

### REFERENCES

Feria-Arroyo, T. P. et al. Implications of climate change on the distribution of the tick vector Ixodes scapularis and risk for Lyme disease in the Texas-Mexico transboundary region. Parasit. Vectors 7, 199 (2014).
Branda, J. A, et al. Advances in Serodiagnostic Testing for Lyme Disease Are at Hand. Clin. Infect. Dis. VIEWPOINTS•CID 2018, 1133
Marques, A. R. Laboratory diagnosis of Lyme disease: advances and challenges. Infect. Dis. Clin. North Am. 29, 295-307 (2015).
CDC Lyme Disease. Available at: https://www.cdc.gov/lyme/index.html.
Trollmo, C., Meyer, A. L., Steere, A. C., Hafler, D. A. & Huber, B. T. Molecular Mimicry in Lyme Arthritis Demonstrated at the Single Cell Level: LFA-1 L Is a Partial Agonist for Outer Surface Protein A-Reactive T Cells. J. Immunol. 166, 5286-5291 (2001).
Thangam, E. B. et al. The Role of Histamine and Histamine Receptors in Mast Cell-Mediated Allergy and Inflammation: The Hunt for New Therapeutic Targets. Front. Immunol, 9, 1873 (2018).
Hatziagelaki, E., Adamaki, M., Tsilioni, I., Dimitriadis, G. & Theoharides, T. C. Myalgic Encephalomyelitis/Chronic Fatigue Syndrome—Metabolic Disease or Disturbed Homeostasis due to Focal Inflammation in the Hypothalamus? J. Pharmacol. Exp. Ther. 367, 155-167 (2018).
Jaiswal, S. et al. CD47 is upregulated on circulating hematopoietic stem cells and leukemia cells to avoid phagocytosis. Cell 138, 271-85 (2009).
Majeti, R. et al. CD47 is an adverse prognostic factor and therapeutic antibody target on human acute myeloid leukemia stem cells. Cell 138, 286-99 (2009).
Lagasse, E. & Weissman, I. L. bcl-2 inhibits apoptosis of neutrophils but not their engulfment by macrophages. J. Exp. Med. 179, 1047-52 (1994).
Willingham, S. B. et al. The CD47-signal regulatory protein alpha (SIRPa) interaction is a therapeutic target for human solid tumors. Proc. Natl. Acad. Sci. U.S.A. 109, 6662-7 (2012).
Weiskopf, K. et al. Engineered SIRPα variants as immunotherapeutic adjuvants to anticancer antibodies. Science 341, 88-91 (2013).
Chao, M. P., Majeti, R. & Weissman, I. L. Programmed cell removal: a new obstacle in the road to developing cancer. Nat. Rev. Cancer 12, 58-67 (2012).
Chao, M. P. et al. Anti-CD47 antibody synergizes with rituximab to promote phagocytosis and eradicate non-Hodgkin lymphoma. Cell 142, 699-713 (2010).
Advani, R. et al. CD47 Blockade by Hu5F9-G4 and Rituximab in Non-Hodgkin's Lymphoma. N. Engl. J. Med. 379, 1711-1721 (2018).
Takimoto, C. H. et al. The Macrophage ‘Do not Eat Me’ Signal, CD47, Is A Clinically Validated Cancer Immunotherapy Target. Ann. Oncol. (2019). doi:10.1093/annonc/mdz006
Sikic, B. I. et al. First-in-Human, First-in-Class Phase I Trial of the Anti-CD47 Antibody Hu5F9-G4 in Patients With Advanced Cancers. J. Clin. Oncol. JCO1802018 (2019). doi:10.1200/JCO.18.02018
Kenedy, M. R., Lenhart, T. R. & Akins, D. R. The role of Borrelia burgdorferi outer surface proteins. FEMS Immunol. Med. Microbiol. 66, 1-19 (2012).

What is claimed is:

1. A method of characterizing an immune response to a pathogen by an individual, the method comprising:
collecting at least one antibody-containing sample from the individual;
contacting said at least one antibody-containing sample from the individual with a diagnostic pathogen;
contacting the diagnostic pathogen with one or more isotype-specific or glycosylation-specific reagents, which reagents are operably linked to a detectable moiety; and
analyzing the diagnostic pathogen for the presence of bound isotype-specific or glycosylation-specific reagents to determine the presence and type of pathogen-specific antibodies, wherein the presence and type is indicative of a pathogen infection and immune response.

2. The method of claim 1, wherein the diagnostic pathogen is an intact pathogen, genetically modified to express a fluorophore.

3. The method of claim 2, wherein the fluorophore is a fluorescent protein.

4. The method of claim 3, wherein the fluorescent protein is selected from the group consisting of green fluorescent protein (GFP), red fluorescent protein (RFP), and analogs thereof.

5. The method of any of claims 1-4, wherein the diagnostic pathogen is a live pathogen or a fixed pathogen.

6. The method of any of claims 1-5, wherein the diagnostic pathogen is a clinical isolate or an environmental isolate.

7. The method of any of claims 1-6, wherein the diagnostic pathogen is from a cell line or cell culture.

8. The method of any of claims 1-7, wherein the diagnostic pathogen is further genetically modified to eliminate expression of proteins and other epitopes that are highly conserved among pathogens.

9. The method of claim 8, wherein the epitopes are present on cell surface proteins.

10. The method of claim 9, wherein the cell surface proteins are flagellar proteins.

11. The method of claim 10, wherein the flagellar proteins are one or both of fliH and flil proteins of Borrelia.

12. The method of any of claims 1-11, wherein the pathogen is a cellular pathogen.

13. The method of claim 12, wherein the pathogen is selected from a bacterium, a fungus, and a protozoan.

14. The method of claim 13, wherein the bacterium is a Spirochaetes.

15. The method of claim 14, wherein the Spirochaetes is a Borrelia sp.

16. The method of any of claims 1-15 wherein the infectious pathogen is a tick-borne pathogen.

17. The method of claim 16, wherein the tick-borne pathogen is Borrelia burgdorferi.

18. The method of any of claims 1-17, wherein the isotype specific reagent is an antibody that recognizes one of IgM, IgG1, IgG2, IgG2a, IgG2b, IgG3, IgG4, IgA, IgA1, IgA2, IgE, IgD or Ig specific glycosylation sites (such as IgA1-G2S1 or IgG4-G0F).

19. The method of claim 18, wherein a cocktail of 2 or more uniquely-labeled isotype/subtype/glycosylation-specific reagents is contacted with the sample.

20. The method of any of claims 1-19, wherein analysis is performed by a method selected from flow cytometry, mass cytometry, sequencing or PCR of sequence barcoded antibodies, and high dimensional/multi-parameter microscopy.

21. The method of any of claims 1-20 wherein the pathogen is a vaccine strain and the presence and type of pathogen-specific antibodies is indicative of response to vaccine immunization.
An additional claim on bead or chip based protein or peptide arrays as the “pathogen”

22. The method of any of claims 1-21, further comprising treating the individual in accordance with the analysis, optionally with one or more of an antihistamine, anti-IgE agent or mast cell stabilizing agent where pathogen-specific IgE antibodies are present.

23. The method of any of claims 1-22, wherein the antibody-containing sample is a blood sample.

24. The method of any of claims 1-23, further comprising monitoring the immune response to the pathogen by the individual for a period of time by repeating a)-d) at a plurality of time points.

25. The method of claim 24, wherein a first antibody-containing sample is collected from the individual at a first time point and a second antibody-containing sample is collected from the individual at a later second time point, wherein detection of increased levels of one or more pathogen-specific antibodies in the second antibody-containing sample compared to the levels of the one or more pathogen-specific antibodies in the first antibody-containing sample indicates that the infection by the pathogen is worsening, and decreased levels of the one or more pathogen-specific antibodies in the second antibody-containing sample compared to the levels of the one or more pathogen-specific antibodies in the first antibody-containing sample indicates that the infection by the pathogen is improving.

26. The method of claim 25, further comprising monitoring the efficacy of a therapy for treating the infection by the pathogen, wherein the first antibody-containing sample is collected from the individual before the patient undergoes the therapy and the second antibody-containing sample is collected from the individual after the patient undergoes the therapy, wherein detection of increased levels of the one or more pathogen-specific antibodies in the second antibody-containing sample compared to the levels of the one or more pathogen-specific antibodies in the first antibody-containing sample indicates that the infection by the pathogen is worsening or not responding to the therapy, and decreased levels of the one or more pathogen-specific antibodies in the second antibody-containing sample compared to the levels of the one or more pathogen-specific antibodies in the first antibody-containing sample indicates that the infection by the pathogen is improving.

27. A diagnostic pathogen for use in the methods of any of claims 1-26.

28. A method of diagnosing and treating an individual with Lyme disease, the method comprising:
collecting at least one antibody-containing sample from the individual;
contacting said at least one antibody-containing sample from the individual with a diagnostic bait displaying a plurality of Borrelia burgdorferi pathogen antigens;
contacting the diagnostic bait with one or more isotype-specific or glycosylation-specific reagents, which reagents are operably linked to a detectable moiety;
analyzing the diagnostic bait for the presence of bound isotype-specific or glycosylation-specific reagents to determine the presence and type of Borrelia burgdorferi pathogen-specific antibodies, wherein the presence and type is indicative of a Borrelia burgdorferi infection and an immune response to the Borrelia burgdorferi pathogen;
diagnosing the individual with Lyme disease if the presence of one or more Borrelia burgdorferi pathogen-specific antibodies is detected, and
treating the individual for Lyme disease if the presence of one or more Borrelia burgdorferi pathogen-specific antibodies is detected, wherein one or more of an antihistamine; anti-IgE agent or mast cell stabilizing agent is administered to the individual if the presence of Borrelia burgdorferi pathogen-specific immunoglobulin E (IgE) antibodies is detected.

29. The method of claim 28, wherein the antihistamine inhibits a histamine receptor selected from the group consisting of H1, H2, H3, and H4.

30. The method of claim 28, wherein the antihistamine is selected from the group consisting of cimetidine, ranitidine, Benadryl, diphenhydramine, loratadine, doxepin, thioperamide, and clobenpropit.

31. The method of any of claims 28-30, further comprising depleting or stabilizing mast cells in the individual if the presence of Borrelia burgdorferi pathogen-specific immunoglobulin E (IgE) antibodies is detected.

32. The method of claim 31, wherein said mast cells are depleted by administering anti-c-kit therapy. I think we should have a list of possible mast cell stabilizing drugs as well and/or refer to the tables in that review

33. The method of claim 32, further comprising administering anti-CD47 therapy.

34. The method of any of claims 28-33, further comprising depleting IgE producing B cells in the individual if the presence of Borrelia burgdorferi pathogen-specific immunoglobulin E (IgE) antibodies is detected.

35. The method of any of claims 28-34, wherein anti-IgE therapy comprises IgE blockade or linkage of IgE specific antibodies to a different isotype with beneficial effector functions.

36. The method of claim 35, wherein the isotype is IgG.

37. The method of any of claims 28-36, further comprising dampening an IgE response by cytokine blockade of one or more cytokines selected from the group consisting of IL-4, IL-5, and IL-13.

38. The method of any of claims 28-37, wherein the diagnostic bait s a diagnostic Borrelia burgdorferi pathogen.

39. The method of claim 38, wherein the diagnostic Borrelia burgdorferi pathogen is genetically modified to express a fluorophore.

40. The method of claim 39, wherein the fluorophore is a fluorescent protein.

41. The method of claim 40 wherein the fluorescent protein is selected from green fluorescent protein (GFP), red fluorescent protein (RFP), and analogs thereof.

42. The method of any of claims 38-41, wherein the diagnostic Borrelia burgdorferi pathogen is a Borrelia burgdorferi clinical isolate or an environmental isolate.

43. The method of any of claims 38-41, wherein the diagnostic Borrelia burgdorferi pathogen is from a Borrelia burgdorferi cell line or cell culture.

44. The method of any of claims 38-43, wherein the diagnostic pathogen is further genetically modified to eliminate expression of proteins and other epitopes that are highly conserved among pathogens.

45. The method of claim 44, wherein the epitopes are present on cell surface proteins.

46. The method of claim 45, wherein the cell surface proteins are fliH or flil proteins of Borrelia or a combination thereof.

47. The method of any of claims 38-46, wherein the diagnostic pathogen is a live pathogen or a fixed pathogen.

48. The method of any of claims 28-47, wherein the diagnostic bait is an antigen array comprising Borrelia burgdorferi pathogen proteins or peptide epitopes.

49. The method of any of claims 28-48, wherein the isotype specific reagent is an antibody that recognizes one of IgM, IgG1, IgG2, IgG2a, IgG2b, IgG3, IgG4, IgA, IgA1; IgA2, IgE, IgD or Ig specific glycosylation sites (such as IgA1-G2S1 or IgG4-G0F).

50. The method of any of claims 28-49, wherein analysis is performed by a method selected from flow cytometry, mass cytometry, sequencing or PCR of sequence barcoded antibodies, and high dimensional/multi-parameter microscopy.

51. The method of any of claims 28-50, further comprising monitoring the immune response to the Borrelia burgdorferi pathogen by the individual for a period of time by repeating at a plurality of time points.

52. The method of claim 51, wherein a first antibody-containing sample is collected from the individual at a first time point and a second antibody-containing sample is collected from the individual at a later second time point, wherein detection of increased levels of one or more Borrelia burgdorferi pathogen-specific antibodies in the second antibody-containing sample compared to the levels of the one or more Borrelia burgdorferi pathogen-specific antibodies in the first antibody-containing sample indicates that the Lyme disease is worsening, and decreased levels of the one or more Borrelia burgdorferi pathogen-specific antibodies in the second antibody-containing sample compared to the levels of the one or more Borrelia burgdorferi pathogen-specific antibodies in the first antibody-containing sample indicates that the Lyme disease is improving.

53. The method of claim 52, further comprising monitoring the efficacy of a therapy for treating Lyme disease, wherein the first antibody-containing sample is collected from the individual before the patient undergoes the therapy and the second antibody-containing sample is collected from the individual after the patient undergoes the therapy, wherein detection of increased levels of the one or more Borrelia burgdorferi pathogen-specific antibodies in the second antibody-containing sample compared to the levels of the one or more Borrelia burgdorferi pathogen-specific antibodies in the first antibody-containing sample indicates that the Lyme disease is worsening or not responding to the therapy, and decreased levels of the one or more Borrelia burgdorferi pathogen-specific antibodies in the second antibody-containing sample compared to the levels of the one or more Borrelia burgdorferi pathogen-specific antibodies in the first antibody-containing sample indicates that the Lyme disease is improving.

54. The method of any of claims 28-53, wherein said treating the individual for Lyme disease further comprises administering an antibiotic.

55. A Borrelia burgdorferi diagnostic pathogen for use in the methods of any of claims 28-47 and 49-54.

56. A kit comprising the Borrelia burgdorferi diagnostic pathogen of claim 55 and one or more isotype-specific or glycosylation-specific reagents for detecting Borrelia burgdorferi pathogen-specific antibodies.

57. The kit of claim 56, wherein the isotype-specific reagents comprise IgE-specific reagents for detecting Borrelia burgdorferi pathogen-specific IgE antibodies.

58. The kit of claim 56 or 57, further comprising an antihistamine or an anti-IgE therapeutic agent.

59. The kit of claim 58, wherein the antihistamine is selected from the group consisting of cimetidine, ranitidine, Benadryl, diphenhydramine, loratadine, doxepin, thioperamide, and clobenpropit.
